(19) United States
(12) Patent Application Publication
(10) Pub. No.: US 2014/0357801 A1
(43) Pub. Date: Dec. 4, 2014

(54) Nitrile rubbers coupled via bisdihydropyrazole groups, production thereof and use thereof

(71) Applicant: LANXESS Deutschland GmbH, Cologne (DE)

(72) Inventors: Sven BRANDAU, Strasbourg (FR); Andreas KAISER, Strasbourg (FR); Uwe WESTEPPE, La Wantzenau (FR); Christopher BARNER-KOWOLLIK, Stutensee-Blankenloch (DE); Christoph DUERR, Heidelberg (DE); Paul LEDERHOSE, Kehl (DE)

(73) Assignee: LANXESS Deutschland GmbH, Cologne (DE)

(21) Appl. No.: 14/283,468

(22) Filed: May 21, 2014

(30) Foreign Application Priority Data
Jun. 3, 2013 (EP) 13170317.5

(51) Int. Cl. C08C 19/22; C07D 257/04

(52) U.S. Cl. CPC C08C 19/22; C07D 257/04
USPC 525/329.3; 525/348; 548/253

(57) ABSTRACT

A nitrile rubber coupled via bisdihydropyrazole groups is produced by reacting a nitrile rubber which is based on conjugated dienes, α,β-unsaturated nitriles and optionally further copolymerizable monomers as monomers, may have been hydrogenated and has covalently bonded tetrazole groups with a bifunctional ene compound in which the ene groups can each be reacted with the tetrazole groups to give dihydropyrazole groups.

### FIELD OF THE INVENTION

The present invention relates to a process for producing nitrile rubbers which have been coupled via bisdihydropyrazole groups and may have been hydrogenated, to rubbers obtainable by the process, to itrile rubbers coupled via tetrazole and ene groups under UV irradiation, to the use thereof, to vulcanizable mixtures and vulcanizates comprising these and to processes for producing the vulcanizates.

### BACKGROUND OF THE INVENTION

More particularly, the present invention relates to the production of nitrite rubbers by free-radical polymerization which is conducted in solution and in the presence of specific chain transfer compounds, with subsequent reaction with bismaleimides under UV irradiation, and also to nitrile rubbers having structural elements originating from the chain transfer compounds in the polymer backbone or at the chain ends.

Nitrile rubbers, also abbreviated to “NBR”, are understood to mean rubbers which are co- or terpolymers of at least one α,β-unsaturated nitrile, at least one conjugated diene and optionally one or more further copolymerizable monomers. Hydrogenated nitrile rubbers (“HNBR”) are understood to mean corresponding co- or terpolymers in which all or some of the C═C double bonds of the copolymerized diene units have been hydrogenated.

For many years, both NBR and HNBR have occupied an established position in the specialty elastomers sector. They possess an excellent profile of properties in the form of excellent oil resistance, good heat stability, excellent resistance to ozone and chemicals, the latter being even more pronounced in the case of HNBR than in the case of NBR. NBR and HNBR also have very good mechanical and performance properties. For this reason, they are widely used in a wide variety of different fields of use, and are used, for example, for production of gaskets, hoses, belts and damping elements in the automotive sector, and also for stators, well seals and valve seals in the oil production sector, and also for numerous parts in the electrical industry, mechanical engineering and shipbuilding. A multitude of different types are commercially available, and these feature, according to the application sector, different monomers, molecular weights, polydispersities and mechanical and physical properties. As well as the standard types, there is increasing demand particularly for specialty types featuring contents of specific termonomers or particular functionalizations.

In the practical use of the (H)NBR rubbers, the vulcanization of the rubbers, i.e. more particularly the crosslinker system and the vulcanization conditions, is also growing in importance. Thus, in addition to the conventional rubber crosslinking systems based on peroxides or sulphur which have already existed for many decades, various new concepts for alternative crosslinking have been developed in the last few years. Crosslinking concepts of this kind also include polymers which, because of functional groups, are not amenable to all forms of crosslinking and crosslinking agents and therefore constitute a particular challenge.

On the industrial scale, nitrile rubbers are produced almost exclusively by what is called emulsion polymerization. To control the molecular weight and hence also the viscosity of the nitrile rubber which forms, it is customary to use dodecyl mercaptans, especially tert-dodecyl mercaptans (“TDDM” or else “TDM”). After the polymerization, the NBR latex obtained is coagulated in a first step and NBR solid is isolated therefrom. If further hydrogenation of the nitrile rubber to give HNBR is desired, this hydrogenation is likewise effected by known prior art methods, for example using homogeneous or else heterogeneous hydrogenation catalysts. The catalysts are typically based on rhodium, ruthenium or titanium. However, it is also possible to use platinum, iridium, palladium, rhenium, ruthenium, osmium, cobalt or copper, either in metal form or else preferably in the form of metal compounds.

There have already been a wide variety of different approaches for optimization of the production processes for NBR and HNBR. For instance, attempts have been made to conduct the polymerization to give the nitrile rubber in organic solution. In the abstract of the thesis by C. Hollbeck, Universität-Gesamthochschule Essen, 1995, page II, the following statement is made in respect of the copolymerization of acrylonitrile and 1,3-butadiene in organic solution (quotation, in translation): “With a number-average degree of polymerization Pn of 1589 (molecular weight (Mn)=˜85 800 g/mol) and a conversion of 40.5%, the objectives set were achieved at a reaction temperature of 343 K within 40 hours. Shortening of the time to 18 hours was only possible with a reduction in the conversion demanded. As test experiments showed, under the given conditions, even with an increase in the temperature to 353 K. a combination of Pn≧1400 and conversion greater than 40% is not within the realm of possibility”. A restriction in the conversion achievable to around 40% within a reaction time of 40 hours makes the process for organic solution polymerization described therein technically and economically unsuitable for practical use.

WO-A-2011/032832 describes the production of nitrile rubbers by free-radical polymerization in the presence of specific chain transfer compounds which are apparent as structural elements in the polymer backbone or at the ends in question.

A corresponding process is also described in Macromol. Rapid Commun., 2010, 31, 1616-21. The solution-based RAFT-mediated synthesis of acrylonitrile-butadiene rubber (NBR) using trithiocarbonates and dithioesters led to molecular weights of up to 60 000 Wmol with PD1 values of <2.0 and a conversion of up to 50% within a reaction time of 9 hours.

Polym. Chem., 2012, 3, 1048 discloses a process for producing a nitrile rubber coupled via bistriazolyl groups. This involves reacting a nitrile rubber having covalently bonded carbon-carbon triple bonds with a diazide, so as to result in bistriazolyi groups in the coupling. The preparation typically requires a metal catalyst, especially a copper catalyst.

The chain transfer compounds described are known from what is called RAFT methodology. This methodology is already used for synthesis of various polymers (WO-A-2001160792, U.S. Pat. No. 7,230,063 B1, WO-A-2007/003782, U8-A-2008/0153982, WO-A-2005/061555).

### SUMMARY OF THE INVENTION

Against the background outlined above, it was thus an object of the present invention firstly to provide nitrile rubbers which enable the formation of particular polymer architectures and microstructures and hence the establishment of particular profiles of properties for the later applications, and also permit simple crosslinking or chain extension. A secondary object which was to be achieved at the same time was that of making these specific nitrile rubbers available with a broad range of molecular weights and polydispersities and via a very simple production process. In addition, a process for producing the polymers in which the reaction times can be minimized while maintaining the molar masses achieved is advantageous for economic reasons. In order to maintain the good ageing properties of the polymers, a process in which no metals are used as catalysts is of interest, since these, as the person skilled in the art is aware, can have adverse effects in this respect.

It has been found that it is possible to produce nitrile rubbers by free-radical polymerization in solution or emulsion using specific RAFT chain transfer agents which allow an increase in the chain lengths by polymer-analogous coupling reactions via tetrazole functionalization.

The product is achieved in accordance with the invention by a process for producing a nitrile rubber coupled via bisdihydropyrazole groups by reacting a nitrile rubber which is based on conjugated dienes, α,β-unsaturated nitriles and optionally further eopolymerizable monomers as monomers, may have been hydrogenated and has covalently bonded tetrazole groups with a bifunctional ene compound in which the ene groups can each be reacted (via a nitrile imine intermediate) with the tetrazole groups to give dihydropyrazole groups and are converted thereto.

The term “coupled via bisdihydropyrazole groups” means coupling by means of a chemical structural element having (at least) two dihydropyrazole groups in chemically bonded form. The dihydropyrazole groups are generally covalently bonded to one another via an organic radical.

### BRIEF DESCRIPTION OF THE DRAWINGS

The appended FIGS. 1 to 3 show the spectrometric and spectroscopic detection of the structures formed in the Examples.

FIG. 1 shows a section from the SEC-mass spectrometry analysis of a tetrazole-functionalized nitrile rubber (NBR 6 in the EXAMPLES). As well as the sodium adducts of the tetrazole-functionalized nitrile rubber A (in the inset, nitrile rubber with 9 repeat units), small amounts of nitrile rubber A* (in the inset, nitrile rubber with 17 repeat units) are also observed, which arise through recombination of two chains with chain ends occupied by initiator fragments.

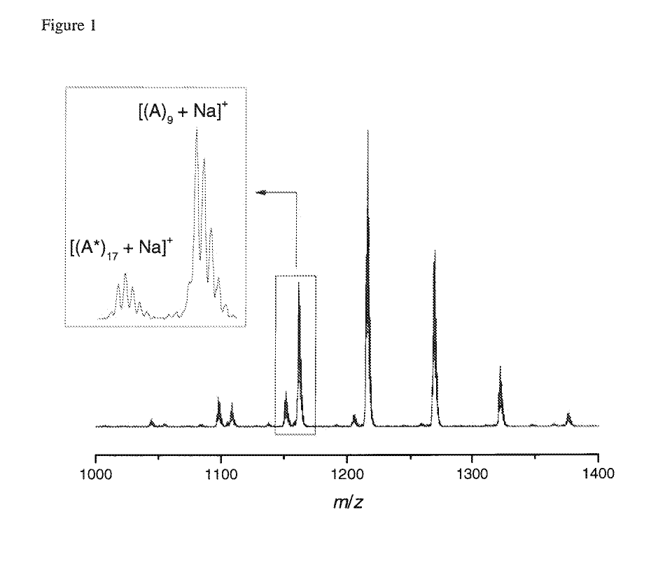

FIG. 2 shows an H NMR spectroscopy analysis of a tetrazole-functionalized nitrile rubber (NBR 6 in the EXAMPLES) and assignment of all the characteristic resonances.

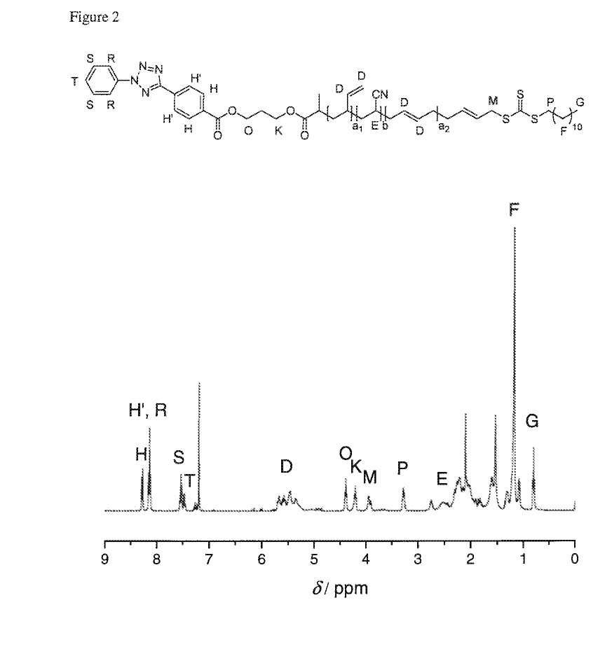

FIG. 3 shows an H NMR spectroscopy comparison of a tetrazole-functionalized nitrile rubber (NBR 2 in the EXAMPLES, top) with the coupled nitrile rubber obtained therefrom by UV irradiation (NBR 8 in the EXAMPLES, bottom).

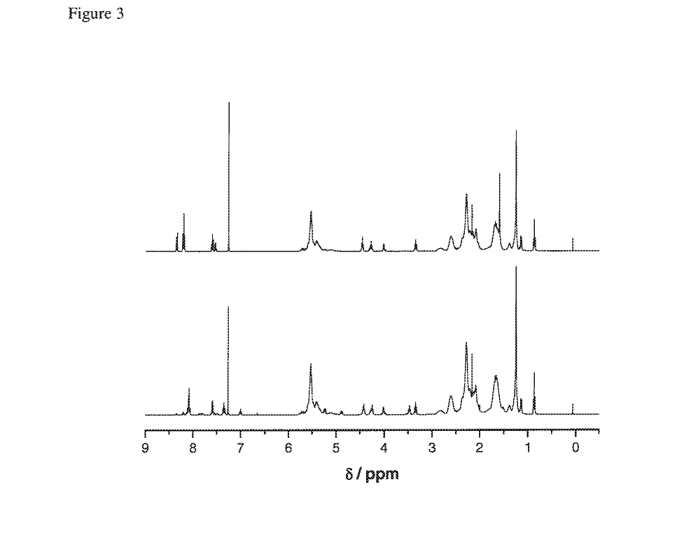

FIG. 4 shows the general synthesis strategy of the examples and, in particular, the UV-induced molecular coupling of the NBR units to form a nitrile rubber having a relatively high molecular weight. The structure shown bottom right in the scheme is supposed to represent the NBR chains.

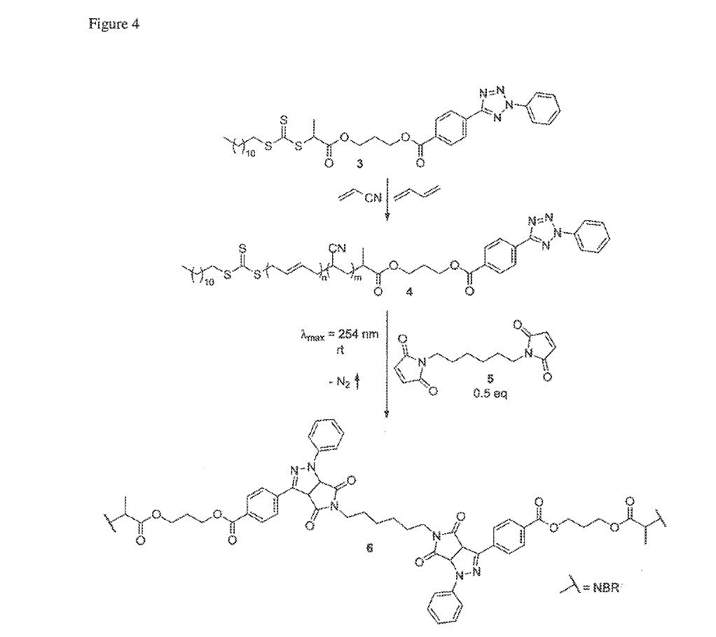

### DETAILED DESCRIPTION OF THE INVENTION

The tetrazole group preferably takes the form of the radical of the general formula (I)

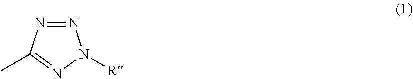

in which R″ is an optionally substituted aryl radical. Suitable aryl radicals are, for example, phenyl, naphthyl or anthracenyl groups, which may be present without substituents or in substituted form. Possible substituents of the aryl radicals are especially C₁-C₁₂-alkyl radicals, more preferably C₁-C₆-alkyl groups, especially C₁-C₃-alkyl groups. For example, a tolyl radical may be present. Possible further substituents of the aryl radicals are amino, carboxyl or hydroxyl functions.

The nitrile rubber to be coupled is preferably obtainable by free-radical polymerization of at least one conjugated diene, at least one α,β-unsaturated nitrile and optionally one or more further copolymerizable monomers, in the presence of at least one organic solvent and at least one chain transfer agent, the chain transfer agent used being at least one compound of the general formula (I)

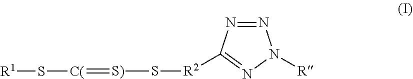

where
R¹ is a hydrocarbyl radical which may be substituted and, after homolytic scission of the R¹—S bond, forms a primary radical,
R² is a hydrocarbyl radical which may be substituted, may contain one or more carboxyl groups and, after homolytic scisson of the S—R² bond, forms a secondary, tertiary or aromatically stabilized radical,
R″ is as specified above.

Preferably, in the compound of the general formula (I), R is a C₈-C₁₅-alkyl radical and R² is a —CHR³C(═O)OR⁴— radical where R³ is C₁-C₃-alkyl and R⁴ is C₁-C₆-alkylene which may be interrupted by carboxyl and/or aryl groups, and R″ is phenyl, tolyl, naphthyl or anthracenyl.

R⁴ may be a C₁-C₆-alkylene radical which may be linear or branched and may be interrupted by carboxyl and/or aryl groups. The carboxyl and/or aryl groups may also be in terminal positions, such that the R⁴ radical is bonded to the tetrazole group via a carboxyl and/or aryl group.

Preferably, the R⁴ radical is bonded to the tetrazole group via an aryl group of this kind. More preferably, this aryl group is bonded to the rest of the R⁴ radical via a carboxyl group. Preferably, the R⁴ radical is a C₁-C₆-alkylene-O—C(═O)-arylene-radical. Arylene is preferably 1,4-phenylene. For R⁴, particular preference is given to C₁₋₆-alkylene, very particular preference to 1,3-propylene.

Preferably, in the bifunctional ene compound, two ene groups of the general formula

R′—X—CH═CH—X—R′ (II)

are present, in which each X is independently a C═O, CHOH, CHI, CHBr, CHCl, CHNO₂, CHNH₂, CHCOOH, CHC₆H₅, CHCN radical and each R′ is independently a C₁-C₁₂-hydrocarbyl radical which may contain one or more heteroatoms, where the two R′ radicals on one ene group of the general formula (II) may be joined to form a ring and the two ene groups of the general formula (II) are covalently bonded to one another via at least one of the R′ radicals in each case.

In an alternative embodiment, in the bifunctional ene compound, two ene groups of the general formula

R′—X—CH═CH—X—R′ (II)

are present, in which each X is independently a C═O, CHOH, CHI, CHBr, CHCl, CHNO₂, CHNH₂, CHCOOH, CHC₆Hc, CHCN radical and each R′ is independently a C₁-C₁₂-hydrocarbyl radical which may contain one or more heteroatoms, wherein the two ene groups of the general formula (II) are covalently bonded to one another via at least one of the R′ radicals in each case or in the alternative either in one or in both ene groups the two R′ radicals of the respective ene group together form a radical Y which represents a C₂-C₂₄-hydrocarbyl radical which may contain one or more heteroatoms, thereby forming a ring together with the adjacent unit —X—CH═CH—X— and wherein the link to the second ene group is then effected by a third coordination valence of the radical Y.

Preferably, the bifunctional ene compound has two maleimide groups wherein the nitrogen atoms are joined to one another via a C₁-C₁₂-alkylene radical which may be substituted and interrupted by heteroatoms and/or aryl groups.

Preferably, the terminal nitrogen atoms are joined to one another via a linear C₄-C₈-alkylene radical.

As an alternative to the above production, the nitrile rubber to be coupled is, in one embodiment of the invention, obtainable by
a) free-radical polymerization of at least one conjugated diene, at least one α,β-unsaturated nitrile and optionally one or more further copolymerizable monomers, preferably in the presence of at least one organic solvent and in the presence of at least one chain transfer agent, and
b) optionally followed by a hydrogenation,
   wherein the chain transfer agent used in step a) is at least one compound of the general structural formula (VI)

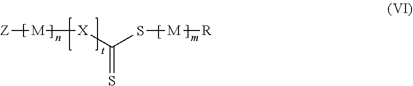

in which
Z is H, a linear or branched, saturated or mono- or polyunsaturated alkyl radical, a saturated or mono- or poly-unsaturated carbo- or hetemcyclyl radical, aryl, heteroaryl, aralkyl, heteroaralkyl, alkoxy, aryloxy, heteroaryloxy, amino, amido, hydroxyimino, carbamoyl, alkoxycarbonyl, F, Cl, Br, I, hydroxyl, phosphonato, phosphinato, alkylthio, arylthio, sulphanyl, thiocarboxyl, sulphinyl, sulphono, sulphino, sulpheno, sulphonic acids, sulphamoyl, silyl, silyloxy, nitrile, carbonyl, carboxyl, oxycarbonyl, oxysulphonyl, oxo, thioxo, borates, selenates, epoxy, cyanates, thiocyanates, isocyanates, thioisocyanates and isocyanides,
(a) if m≠0 has the same meanings as the Z radical and
   (b) if m=0 is H, a linear or branched, saturated or mono- or polyunsaturated alkyl radical, a saturated or mono- or polyunsaturated carbo- or hetemcyclyl radical, aryl, heteroaryl, aralkyl, heteroaralkyl, alkoxy, aryloxy, heteroaryloxy, amino, amido, carbamoyl, alkoxy, aryloxy, alkylthio, arylthio, sulphanyl, thiocarboxyl, sulphinyl, sulphono, sulphino, sulpheno, sulphonic acids, sulphamoyl, carbonyl, carboxyl, oxycarbonyl, oxysulphonyl, oxo, thioxo, epoxy, cyanates, thiocyanates, isocyanates, thioisocyanates or isocyanides,
M represents repeat units of one or more mono- or polyunsaturated monomers comprising conjugated or non-conjugated dienes, alkynes and vinyl compounds, or a structural element which derives from polymers comprising polyethers, especially polyalkylene glycol ethers and polyalkylene oxides, polysiloxanes, polyols, polycarbonates, polyurethanes, poiyisocyanates, polysaccharides, polyesters and polyamides,
n and m are the same or different and are each in the range from 0 to 10 000,
t is 0 or 1 if n=0, and is 1 if n≠0, and
X is C(Z₂), N(Z), P(Z), P(═O)(Z), O, S, S(═O) or S(═O)₂, where Z in these radicals has the same meanings as stated above for the formula (VI),
   and subsequent reaction of the nitrile rubber thus obtained with a compound which allows covalent attachment of a tetrazole group, preferably of a radical of the general formula (I) in which R″ is any aryl group, and leads to this attachment.

The invention relates further to a nitrile rubber coupled via dihydropyrazole groups, preferably obtainable by the above process.

The invention relates additionally to the use of the nitrile rubber coupled via dihydropyrazole groups for production of mouldings, coatings or vulcanizates.

The invention relates additionally to vulcanizable mixtures comprising the above-described nitrile rubber, at least one crosslinker, optionally at least one filler and optionally one or more further rubber additives.

The invention relates additionally to a process for producing vulcanizates, in which the vulcanizable mixture described is subjected to crosslinking, preferably by addition of at least one crosslinker or by photochemical activation.

The invention relates further to vulcanizates, preferably mouldings, obtainable by the above process.

The invention relates further to a chain transfer agent of the general formula (I) as defined above.

The invention relates further to a nitrile rubber which has tetrazole groups and is suitable for coupling with ene groups under UV irradiation, as defined above.

With the process according to the invention, it is possible to arrive at conversions which make the process suitable for an industrial scale reaction within periods comparable to those for the conventional emulsion polymerization for NBR production. For instance, in a polymerization time of less than 10 hours, it is already possible to achieve a conversion of 50% with simultaneous production of industrially acceptable molecular weights (Mn>50 000 g/mol) and with—compared to conventional emulsion NBR—low polydispersities of much less than 2.0 which have not been achieved to date.

It has been found in accordance with the invention that nitrile rubbers having tetrazole functions can be coupled to organic compounds having at least two ene groups, such as bismaleimides, to form dihydropyrazole groups.

Preferably, nitrile rubbers are obtained by free-radical polymerization of the starting monomers in the presence of a chain transfer agent containing a tetrazole group. Particular preference is given to using tetrazole-functionalized trithiocarbonates for this purpose.

Through the use of these tetrazole-functionalized trithiocarbonates as RAFT chain transfer agents, it was possible to obtain a-functionalized NBR units. Preferably, the preparation was effected in the presence of azo initiators in organic solvents such as chlorobenzene or acetone. It was then possible to use these a-functionalized NBR units in the preferably UV-induced tetrazole ene coupling reaction with linking reagents such as 1,6-bis(maleimido)hexane. The polymer-polymer coupling led to linear polymers having molecular weights in the range from especially 8900 g/mol to 94 000 g/mol and polydispersities in the range from especially 1.3 to 1.7.

By means of the UV-induced tetrazole ene reaction, it is possible to distinctly increase the chain length of NBR rubbers and to arrive at high molecular weights.

In the context of this application, the term “nitrile rubber(s)” should be interpreted broadly and encompasses both the nitrile rubbers and hydrogenated nitrile rubbers. If the nitrile rubbers are hydrogenated nitrile rubbers, the abovementioned wording “nitrile rubbers containing repeat units derived from” thus means that the repeat units based on the conjugated diene are those in which the C═C double bonds present at first in the polymer after the polymerization have been fully or partly hydrogenated.

Where this application uses the term “substituted”, this means that a hydrogen atom on a given radical or atom has been replaced by one of the groups specified, with the proviso that the valency of the given atom is not exceeded, and only ever under the condition that this substitution leads to a chemically stable compound.

In the difunctional ene compound (in the ene groups of the general formula (II)), possible substituents are ones that do not hinder the reaction of the difunctional ene compound with the tetrazole radical in the coupling reaction. Examples of possible substituents are given above.

The ene compound may also be substituted by further ene groups. This gives polyene-functional compounds bearing more than two ene functionalities. These are thus organic compounds bearing at least two ene functionalities.

In the compounds of the general formula (I) used with preference as chain transfer agents, it is also possible for the R¹ and R², and also R″, radicals to be substituted. These substituents too are selected such that they do not restrict the tetrazole ene reaction and chemical stability of the compound of the general formula (I). Preferably, substituents of the R¹ and R², and also R″, radicals are C₁-C₆-alkyl radicals.

Preferably, in the difunctional ene compound, electron-withdrawing substituents are present, preferably selected from OH, ═O, —I, —Br, —Cl, —NO₂, —NH₂, —COOH, —C₆H₅, —CN.

The NBR having a covalently bonded tetrazole function contains a tetrazole function suitable for conversion in the coupling reaction described. The steric arrangement and possible attachment groups thereof are selected such that the UV-activated coupling reaction is promoted.

More preferably, the tetrazole group is covalently attached in a terminal position at the chain end or in a side chain of the NBR and has the structure of the general formula (1) where R″ is benzyl or any aromatic system. Preferably, R″ is phenyl or benzyl.

In the reaction between ene functionalities of the at least difunctional ene compound and tetrazole functionalities, preference is given to setting such a molar ratio as to approximate very closely to a molar ratio between ene functionalities and tetrazole functionalities of 1:1. Preferably, the molar ratio is 1:0.5 to 0.5:1, more preferably 1:0.8 to 0.8:1, particularly 0.94:1 to 1:0.94, especially 0.99:1 to 1:0.99.

The more exactly the amounts of tetrazole groups and ene functionalities are matched to one another, the higher the molecular weights that are attainable. When there is a comparatively significant deviation from the equimolar ratio, the result is a comparatively broad molecular weight distribution.

The conversion is preferably performed in an organic solvent, more preferably in an organic polar solvent that dissolves NBR. Particularly preferred solvents are DMF, pyridine, methylene chloride, acetonitrile or acetone, and the further solvents mentioned below for the NBR production.

If a hydrogenation of the NBR is performed after the formation of the dihydropyrazole, it is preferable to use the same solvent for the coupling as for the hydrogenation.

For the coupling, the NBR is present preferably in a concentration of 0.01 to 100 g/l, more preferably 0.1 to 60 g/l, especially 0.6 to 20 g/l.

The coupling can generally be effected within broad concentration ranges and at any suitable temperature. The coupling is preferably performed at a temperature in the range from 0 to 200° C., more preferably 20 to 140° C., especially about room temperature (22-28° C.). The coupling can generally be effected under air.

The coupling is preferably effected with incidence of electromagnetic waves. These enable the absence of any metallic catalyst. Because of the use properties, it is advantageous to conduct the coupling without metal, since metal contents can worsen the use properties of the NBR obtained.

The solution of the tetrazole-functional NBR and of the ene-functional coupling reagent is exposed to electromagnetic radiation. This radiation preferably has a wavelength in the range from 10 to 800 nm, more preferably 100 to 400 nm. This wavelength is most preferably within the range from 200 to 380 nm.

The process according to the invention allows high molecular weights to be achieved in the NBR within short reaction times, coupled with a narrow PDI. Preferably, the nitrile rubbers coupled via dihydropyrazole groups have a molecular weight (M_{w}) in the range from 2000 to 500 000 g/mol, more preferably 5000 to 400 000 g/mol. The polydispersity index (PDI) is preferably less than 2.0, more preferably 1.7 or less. especially 1.3 to 1.7.

The nitrile rubber having covalently bonded tetrazole functionalities which is used for coupling can be obtained by different processes. For example, the unfunctionalized nitrite rubber can first be prepared using a known RAFT chain transfer agent, and the radical of the general formula (1) is subsequently attached by reaction with a suitable compound which has this moiety and enables attachment to the nitrile rubber. This form of reaction can be conducted under conditions familiar to those skilled in the art. Alternatively, the tetrazole functionality can be introduced by methods familiar to those skilled in the art into the RAFT chain transfer agent, which is then used during the polymerization to establish the desired molecular weight and thus incorporated into the polymer chain.

In the first method, nitrile rubbers are first produced by
a) conducting a free-radical polymerization of at least one conjugated diene, at least one α,β-unsaturated nitrile and optionally one or more further copolymerizable monomers in the presence of at least one organic solvent and at least one chain transfer agent and
b) optionally a subsequent hydrogenation,
   wherein the chain transfer agent used is at least one compound of the general structural formula (VI)

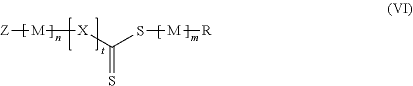

in which
Z is H, a linear or branched, saturated or mono- or polyunsaturated alkyl radical, a saturated or mono- or polyunsaturated carbo- or heterocyclyl radical, aryl, heteroaryl, aralkyl, heteroaralkyl, alkoxy, aryloxy, heteroaryloxy, amino, amido, hydroxyimino, carbamoyl, alkoxycarbonyl, F, Cl, Br, I, hydroxyl, phosphonato, phosphinato, alkylthio, arylthio, sulphanyl, thiocarboxyl, sulphinyl, sulphono, sulphino, sulpheno, sulphonic acids, sulphamoyl, silyl, silyloxy, nitrile, carbonyl, carboxyl, oxycarbonyl, oxysulphonyl, oxo, thioxo, borates, selenates, epoxy, cyanates, thiocyanates, isocyanates, thioisocyanates and isocyanides,
R (a) if m≠0 can have the same meanings as the Z radical and
   (b) if m=0 is H, a linear or branched, saturated or mono- or polyunsaturated alkyl radical, a saturated or mono- or polyunsaturated carbo- or heterocyclyl radical, aryl, heteroaryl, aralkyl, heteroaralkyl, alkoxy, aryloxy, heteroaryloxy, amino, amido, carbamoyl, alkoxy, aryloxy, alkylthio, arylthio, sulphanyl, thiocarboxyl, sulphinyl, sulphono, sulphino, sulpheno, sulphonic acids, sulphamoyl, carbonyl, carboxyl, oxycarbonyl, oxysulphonyl, oxo, thioxo, epoxy, cyanates, thiocyanates, isocyanates, thioisocyanates or isocyanides,
M represents repeat units of one or more mono- or polyunsaturated monomers comprising conjugated or non-conjugated dienes, alkynes and vinyl compounds, or a structural element which derives from polymers comprising polyethers, especially polyalkylene glycol ethers and polyalkylene oxides, polysiloxanes, polyols, polycarbonates, polyurethanes, polyisocyanates, polysaccharides, polyesters and polyamides,
n and m are the same or different and are each in the range from 0 to 10 000,
t is 0 or 1 if n=0, and is 1 if n≠0, and
X is C(Z₂), N(Z), P(Z), P(═O)(Z), O, S, S(═O) or S(═O)₂, where Z in these radicals can have the same meanings as stated above for the formula (VI).

The inventive, optionally hydrogenated nitrile rubbers are notable for the presence of one or more structural elements of the general formulae (I) or (VI), either in the polymer backbone or as end groups.

The meanings specified in the Z and R radicals of the general formula (VI) may each be mono- or polysubstituted. Preferably, the following radicals may have mono- or polysubstituted: alkyl, carbocyclyl, heterocyclyl, aryl, heteroaryl, aralkyl, heteroaralkyl, alkoxy, aryloxy, alkylthio, arylthio, amino, amido, carbamoyl, phosphonato, phosphinato, sulphanyl, thiocarboxyl, sulphinyl, sulphono, sulphino, sulpheno, sulphamoyl, silyl, silyloxy, carbonyl, carboxyl, oxycarbonyl, oxysulphonyl, oxo, thioxo, borates, selenates and epoxy.

Possible substituents again include—provided that chemically stable compounds are the result—all the meanings that Z can assume. Particularly suitable substituents are halogen, preferably fluorine, chlorine, bromine or iodine, nitrile (CN) and carboxyl.

The meanings specified for Z and R in the general formula (VI) explicitly also include salts of the radicals mentioned, provided that they are chemically possible and stable. These may be, for example, ammonium salts, alkali metal salts, alkaline earth metal salts, aluminium salts or protonated forms of the chain transfer agents of the general formula (VI).

The meanings specified for Z and R in the general formula (VI) also include organometallic radicals, for example those that impart a Grignard functionality to the chain transfer agent. Z and R may also be or have a carbanion, with lithium, zinc, tin, aluminium, lead and boron as counterion.

It is additionally possible that the chain transfer agent is coupled via a linker to a solid phase or carrier substance. The linker may be the Wang, Sasrin, Rink acid, 2-chlorotrityl, Mannich, safety-catch, traceless or photolabile linker known to those skilled in the art. Useful solid phases or carrier substances include, for example, silica, ion exchange resins, clays, montmorillonites, crosslinked polystyrene, polyethylene glycol grafted onto polystyrene, polyacrylamides (“Pepsyn”), polyethylene glycol acrylamide copolymers (PEGA), cellulose, cotton and controlled pore glass (CPG).

It is additionally possible that the chain transfer agents of the general formula (VI) function as ligands for organometallic complexes, for example for those based on the central metals rhodium, ruthenium, titanium, platinum, iridium, palladium, rhenium, ruthenium, osmium, cobalt, iron or copper.

The meanings listed for the “M” radical in the abovementioned general formula (VI) may be mono- or polysubstituted. Thus, M may comprise repeat units of one or more mono- or polyunsaturated monomers, preferably optionally mono- or polysubstituted conjugated or non-conjugated dienes, optionally mono- or polysubstituted alkynes or optionally mono- or polysubstituted vinyl compounds, for example fluorinated mono- or polyunsaturated vinyl compounds, or else a divalent structural element which derives from substituted or unsubstituted polymers including polyethers, especially polyalkylene glycol ethers and polyalkylene oxides, polysiloxanes, polyols, polycarbonates, polyurethanes, polyisocyanates, polysaccharides, polyesters and polyamides. Behind these “M” radicals may thus be a monomeric or polymeric radical.

Preference is given to using a chain transfer agent of the general formula (VI) in which
Z and R have the meanings specified above for the general formula (VI) and
n, m and t are all equal to zero.

This preferred chain transfer agent thus has the general structure (VIa):

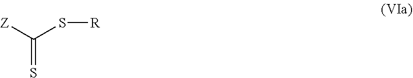

in which the Z and R radicals may have all the meanings specified above for the general formula (VI).

Trithiocarbonates:

As a further preferred chain transfer agent, it is possible to use a chain transfer agent of the general formula (VIb)

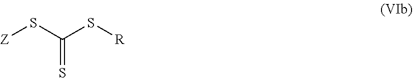

in which
Z has the meanings specified above for the general formula (VI),
R has the meanings specified above for the general formula (VI) for variant b) when m=0, but with the restriction that R, after homolytic scission of the S—R bond, forms a secondary, tertiary or aromatically stabilized radical.

This particularly preferred chain transfer agent of the general formula (VIb) is the chain transfer agent of the general formula (VI) where
n and m are each=0,
t is 1,
X is sulphur,
Z has the meanings specified above for the general formula (VI) and
R has the meanings specified above for the general formula (VI) for variant b) when m=0, but with the restriction that R, after homolytic scission of the S—R bond, forms a secondary, tertiary or aromatically stabilized radical.

These particularly preferred chain transfer agents of the general formula (VIb), depending on whether or not Z and R are identical within the scope of the above definitions, are thus symmetric or asymmetric trithiocarbonates.

Particular preference is given to using a chain transfer agent of the general formula (VIb) in which
Z has the meanings specified above for the general formula (VI) and
R, with the proviso that R, after homolytic scission of the S—R bond, forms a secondary, tertiary or aromatically stabilized radical, is
   a linear or branched, saturated or mono- or polyunsaturated, optionally mono- or polysubstituted alkyl radical, preferably a corresponding C₃-C₂₀-alkyl radical, especially sec-butyl, tert-butyl, isopropyl, 1-buten-3-yl, 2-chloro-1-buten-2-yl, (propionic acid)-2-yl, (propionitrile)-2-yl, (2-methylpropanenitrile)-2-yl, (2-methylpropionic acid)-2-yl or 1H,1H,2-keto-3-oxo-4H,4H,5H,5H-perfluoroundecanyl, or
   a saturated or mono- or polyunsaturated, optionally mono- or polysubstituted carbo- or heterocyclyl radical, especially cyclohexyl, cumyl or (cyclohexane-1-nitrile)-1-yl,
   a (hetem)aryl radical, most preferably a C₆-C₂₄-(hetero)aryl radical, especially phenyl, pyridinyl or withracenyl,
   a (hetero)aralkyl radical, most preferably benzyl, phenylethyl or 1-methyl-1-phenyleth-2-yl, or
   thiocarboxyl, carbonyl, carboxyl, oxo, thioxo, epoxy, or else salts of the aforementioned compounds.

Especially preferably, in addition, a chain transfer agent of the general formula (VIb) is used, in which
Z has the meanings specified above for the general formula (VI), but also with the additional restriction to those definitions such that Z, after homolytic scission of the Z—S bond, forms a secondary, tertiary or aromatically stabilized radical.

In that case, this is a trithiocarbonate chain transfer agent in which both the R and Z radicals have polymerization-initiating action.

Most preferably, in addition, a chain transfer agent of the general formula (VIb) is used, in which
R and Z are the same or different and, with the proviso that R and Z, after homolytic scission of the R—S or the Z—S bond, each form a secondary, tertiary or aromatically stabilized radical, are each
   a linear or branched, saturated or mono- or polyunsaturated, optionally mono- or polysubstituted alkyl radical, preferably a corresponding C₃-C₂₀-alkyl radical, especially sec-butyl, tert-butyl, isopropyl, 1-buten-3-yl, (2-chloro-1-buten-2-yl, (propionic acid)-2-yl, (propionitrile)-2-yl, (2-methylpropanenitrile)-2-yl, (2-methylpropionic acid)-2-yl or 1H,1H,2-keto-3-oxo-4H,4H,5H,58-perfluoroundecanyl, or
   a saturated or mono- or polyunsaturated, optionally mono- or polysubstituted carbo- or heterocyclyl radical, especially cyclohexyl, cumyl or (cyclohexane-1-nitrile)-1-yl,
   a (hetero)aryl radical, most preferably a C₆-C₂₄-(hetero)aryl radical, especially phenyl, pyridinyl or anthracenyl,
   a (hetero)aralkyl radical, most preferably benzyl, phenylethyl or 1-methyl-1-phenyleth-2-yl, or
   thiocarboxyl, carbonyl, carboxyl, oxo, thioxo, epoxy, or else salts of the aforementioned compounds.

With regard to the forms of words “that R, after hemolytic scission of the R—S bond, forms a secondary or tertiary radical” used for the general formula (VIb) and subsequently for the general formulae (VIc), (VId) and (VIe), the definitions which follow apply. These likewise apply analogously to the corresponding form of words “that Z, after homolytic scission of the Z—S bond, forms a secondary or tertiary radical”, where this is used in connection with Z in the context of the application.

The atom in the R radical that implements the bond to S in the general formula (VIb) (or the general formulae (VIc), (VId) and (VIe) still to follow) leads, on homolytic scission of the R—S bond, to a radical which can be described as “tertiary” when at least the following are bonded to this atom (excluding the bond to sulphur):

(i) three substituents via single bonds or
(ii) one substituent via a single bond and a further substituent via a double bond or
(iii) one substituent via a triple bond,
where all the aforementioned substituents must be different from hydrogen.

The atom in the R radical that implements the bond to S in the general formulae (VIb), (VIc), (VId) and (VIe) leads, on homolytic scission of the R—S bond, to a radical which can be described as “secondary” when the following are bonded to this atom:

(i) two substituents via single bonds or
(ii) one substituent via a double bond,
where all the aforementioned substituents must be different from hydrogen and all the further possible substituents are H.

Examples of R or Z radicals which, on homolytic scission of the R—S (or Z—S) bond, lead to a radical which can be described as “tertiary” are, for example, tert-butyl, (cyclohexane-1-nitrile)-1-yl and (2-methylpropanenitrile)-2-yl.

Examples of R or Z radicals which, on homolytic scission of the R—S (or Z—S) bond, lead to a radical which can be described as “secondary” are, for example, sec-butyl, isopropyl and cycloalkyl, preferably cyclohexyl.

With regard to the proviso used hereinafter for the formula (VId), “that Z, after homolytic scission of the Z—S bond, forms a primary radical”, the following definition applies: the atom in the Z radical that implements the bond to S in the general formula (VId) leads, on homolytic scission of the Z—S bond, to a radical which can be described as “primary” where no non-hydrogen substituent or a maximum of one non-hydrogen substituent is bonded to this atom via a single bond. When Z═H, the abovementioned proviso is fulfilled by definition.

Examples of Z radicals that lead, on homolytic scission of the Z—S bond, to a radical which can be described as “primary” are thus, for example, H, linear C₁-C₂₀ alkyl radicals, OH, SH, SR and C₂-C₂₀ alkyl radicals having branches beyond the carbon atom that implements the bond to S.

### Dithioesters:

As a further preferred chain transfer agent, it is possible to use a chain transfer agent of the general formula (VIc)

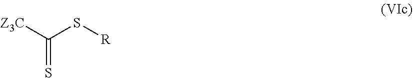

Z has the meanings specified above for the general formula (VI),
R has the meanings specified above for the general formula (VI) for variant b) when m=0, but with the restriction that R, after homolytic scission of the S—R bond, forms a secondary, tertiary or aromatically stabilized radical.

This particularly preferred chain transfer agent of the general formula (VIc) is the chain transfer agent of the general formula (VI) where
n and m are each=0,
t is 1,
X is C(Z)₂,
Z has the meanings specified above for the general formula (VI) and
R has the meanings specified above for the general formula (VI) for variant b) when m=0, but with the restriction that R, after homolytic scission of the S—R bond, forms a secondary, tertiary or aromatically stabilized radical.

Particular preference is given to using a chain transfer agent of the general formula (VIc) in which
R, with the proviso that R, after homolytic scission of the S—R bond, forms a secondary, tertiary or aromatically stabilized radical, is
   a linear or branched, saturated or mono- or polyunsaturated, optionally mono- or polysubstituted alkyl radical, preferably a corresponding C₃-C₂₀-alkyl radical, especially sec-butyl, tert-butyl, isopropyl, 1-buten-3-yl, 2-chloro-1-buten-2-yl, (propionic acid)-2-yl, (propionitrile)-2-y, (2-methylpropanenitrile)-2-yl, (2-methylpropionic acid)-2-yl or 1H, 1H,2-keto-3-oxo-41H,4H,5H,5H-perfluoroundecanyl, or
   a saturated or unsaturated, optionally mono- or polysubstituted carbo- or heterocyclyl radical, especially cyclohexyl, cumyl or (cyclohexane-1-nitrile)-1-yl,
   a (hetero)aryl radical, most preferably a C₆-C₂₄-(hetero)aryl radical, especially phenyl, pyridinyl or anthracenyl,
   a (hetero)arylalkyl radical, most preferably a C₇-C₂₅-(hetero)arylalkyl radical, especially benzyl, phenylethyl or 1-methyl-1-phenyleth-2-yl, or
   thiocarboxyl, carbonyl, carboxyl, oxo, thioxo, epoxy, or else salts of the aforementioned compounds.

### Asymmetric Trithiocarbonates:

In a further preferred embodiment, at least one chain transfer agent of the general formula (VId)

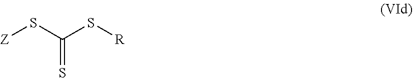

is used, in which
Z has the meanings specified above for the general formula (VI), but with the restriction that Z, after homolytic scission of the S—Z bond, forms a primary radical, and
R may have the same meanings as Z in the general formula (VI), but with the restriction that R, after homolytic scission of the S—R bond, forms a secondary, tertiary or aromatically stabilized radical, and
   with the additional proviso that Z and R assume different meanings.

This preferred chain transfer agent of the general formula (VId) is the chain transfer agent of the general formula (VI) where
n and m are each=0,
t is 1.
X is sulphur,
Z has the meanings specified above for the general formula (VI), but with the restriction that Z, after homolytic scission of the S—Z bond, forms a primary radical, and
R may have the same meanings as Z in the general formula (VI), but with the restriction that R, after homolytic scission of the S—R bond, forms is a secondary, tertiary or aromatically stabilized radical.

These particularly preferred chain transfer agents of the general formula (VId) are thus asymmetric trithiocarbonates.

Particular preference is given to using a chain transfer agent of the abovementioned general formula (VId), in which
Z, with the proviso that Z, after homolytic scission of the S—Z bond, forms a primary radical, is H, a linear or branched, saturated or mono- or polyunsaturated, optionally mono- or polysubstituted alkyl radical, most preferably a corresponding C₁-C₁₆-alkyl radical, especially methyl, ethyl, n-prop-1-yl, but-2-en-1-yl, n-pent-1-yl, n-hex-1-yl or n-dodecan-1-yl, aralkyl, most preferably C₇-C₂₅-aralkyl, especially benzyl, amino, amido, carbamoyl, hydroxyimino, alkoxy, aryloxy, F, Cl, Br, I, hydroxyl, alkylthio, arylthio, carbonyl, carboxyl, oxo, thioxo, cyanates, thiocyanates, isocyanates, thioisocyanates, isocyanides or salts of the compounds mentioned and
R, with the proviso that R, after homolytic scission of the S—R bond, forms a secondary, tertiary or aromatically stabilized radical, is
   a linear, branched or cyclic saturated or mono- or polyunsaturated, optionally mono- or polysubstituted alkyl radical, preferably a corresponding C₃-C₂₀-alkyl radical, especially sec-butyl, tert-butyl, isopropyl, 1-buten-3-yl, 2-chloro-1-buten-2-yl, (propionic acid)-2-yl, (propionitrile)-2-yl, (2-methylpropanenitrile)-2-yl, (2-methylpropionic acid)-2-yl or 1H, 1H,2-keto-3-oxo-4H,4H,5H,5H-perfluoroundecanyl, or
   a saturated or unsaturated, optionally mono- or polysubstituted carbo- or heterocyclyl radical, especially cyclohexyl, cumyl or (cyclohexane-1-nitrile)-1-yl,
   a (hetero)aryl radical, most preferably a C₆-C₂₄-aryl radical, especially phenyl, pyridinyl or anthracenyl,
   an aralkyl radical, most preferably benzyl, phenylethyl or 1-methyl-1-phenyleth-2-yl, or
   thiocarboxyl, carbonyl, carboxyl, oxo, thioxo, epoxy, or else salts of the aforementioned compounds.

### Dithioesters:

In a further preferred embodiment, at least one chain transfer agent of the general formula (VIe)

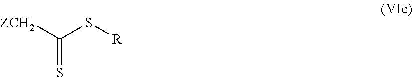

is used, in which
Z may have all the meanings specified for the general formula (VI) and
R may have the same meanings as Z in the general formula (VI), but with the restriction that R, after homolytic scission of the S—R bond, forms a secondary, tertiary or aromatically stabilized radical.

This preferred chain transfer agent of the general formula (VIe) is the chain transfer agent of the general formula (VI) where
n and in are each 0,
t is 1,
X is CH₂,
Z has the meanings specified above for the general formula (VI) and
R may have the same meanings as Z in the general formula (VI), but with the restriction that R, after homolytic scission of the S—R bond, forms a secondary, tertiary or aromatically stabilized radical.

Particular preference is given to using a chain transfer agent of the abovementioned general formula (VIe) in which
R, with the proviso that R, after homolytic scission of the S—R bond, forms a secondary, tertiary or aromatically stabilized radical, is
   a linear or branched, saturated or mono- or polyunsaturated, optionally mono- or polysubstituted alkyl radical, preferably a corresponding C₃-C₂₀-alkyl radical, especially sec-butyl, tert-butyl, isopropyl, 1-buten-3-yl, 2-chloro-1-buten-2-yl, (propionic acid)-2-yl, (propionitrile)-2-yl, (2-methylpropanenitrile)-2-yl, (2-methylpropionic acid)-2-yl or 1H,1H,2-keto-3-oxo-4H,4H,5H,5H-perfluoroundecanyl, or
   a saturated or unsaturated, optionally mono- or polysubstituted carbo- or heterocyclyl radical, especially cyclohexyl, cumyl or (cyclohexane-1-nitrile)-1-yl,
   a (hetero)aryl radical, most preferably a C₆-C₂₄-(hetero)atyl radical, especially phenyl, pyridinyl or anthracenyl,
   a (hetero)arylalkyl radical, most preferably most preferably a C₇-C₂₅-(hetero)arylalkyl radical, especially benzyl, phenylethyl or 1-methyl-1-phenyleth-2-yl, or
   thiocarboxyl, carbonyl, carboxyl, oxo, thioxo, epoxy, or else salts of the aforementioned compounds.

All the aforementioned chain transfer agents are synthesizable by methods familiar to those skilled in the art from the prior art. Synthesis methods and further references to preparation methods can be found, for example, in Polymer 49 (2008) 1079-1131 and in all the property rights and literature references already cited in this application as prior art. A number of the chain transfer agents are also already commercially available.

Particularly suitable chain transfer agents for the process according to the invention are dodecylpropanoic acid trithiocarbonate (DoPAT), dibenzoyl trithiocarbonate (DiBenT), cumylphenyl dithioacetate (CPDA), cumyl dithiobenzoate, phenylethyl dithiobenzoate, cyanoisopropyl dithiobenzoate, 2-cyanoethyl dithiobenzoate, 2-cyanoprop-2-yl dithiophenylacetate, 2-cyanoprop-2-yl dithiobenzoate, S-thiobenzoyl-1H, H,2-keto-3-oxa-4H,4H,5H,5H-perfluoroundecanethiol and S-thiobenzoyl-1-phenyl-2-keto-3-oxa-4H,4H,5H,511-perfluoro-undecanethiol.

Typically, 1 to 2000 mol % of the chain transfer agent are used here, based on 1 mol of the initiator. Preferably, 2 to 1000 mol % of the chain transfer agent are used, based on 1 mol of the initiator, more preferably 4 to 100 mol %.

Preferably in accordance with the invention, the nitrile rubber to be coupled is obtained by free-radical polymerization of at least one conjugated diene, at least one α,β-unsaturated nitrite and optionally one or more further copolymerizable monomers, in the presence of at least one organic solvent and at least one chain transfer agent, the chain transfer agent used being at least one compound of the general formula (I)

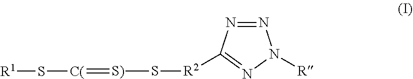

where
R¹ is a hydrocarbyl radical, for example C₅-C₂₀-hydrocarbyl radical which may be substituted and, after homolytic scission of the R¹—S bond, forms a primary radical,
R² is a hydrocarbyl radical, for example C₁-C₁₂-hydrocarbyl radical which may be substituted, may contain one or more carboxyl groups and, after homolytic scission of the S—R² bond, forms a secondary, tertiary or aromatically stabilized radical,
R″ is any aromatic system, for example phenyl, benzyl.

For the definition of possible substituents, of primary radicals, and of secondary, tertiary or aromatically stabilized radicals, reference may be made to the above remarks.

Preferably, in the compounds of the general formula (I), R¹ is a C₈-C₁₅-alkyl radical, especially C₁₀-C₁₄-alkyl radical, especially C₁₁-C₁₃-alkyl radical. Particular preference is given to linear alkyl radicals.

R² is preferably a —CHR³—C(═COOR⁴-radical where R³ is C₁-C₃-alkyl, more preferably methyl or ethyl, especially methyl, and R⁴ is a C₁-C₆-alkylene radical which may be interrupted by carboxyl and/or aryl groups. R″ is preferably phenyl, tolyl, naphthyl or anthracenyl.

This embodiment of the invention has the advantage that high molecular weights are obtainable with use of small amounts of initiator, and very high end group trueness is achieved, meaning that, in the polymer molecules obtained, a high proportion of tetrazole end groups is present. Based on the initiator, the chain transfer agents of the general formula (I) are preferably used in an amount of 5 to 2000 mol %, more preferably 20 to 1500 mol %, especially 500 to 1500 mol %. This means, more particularly, a 4- to 14-fold molar excess of RAFT chain transfer agent of the general formula (I). The high end group trueness means, secondly, a high functionality density, such that a high proportion of tetrazole groups, based on the polymer chains, is present.

The initiators and monomers, and also solvents, suitable for the reaction with the chain transfer agent of the general formula (F) are listed hereinafter. These relate to the preparation of the NBRs with the chain transfer agent of the general formula (VI).

### Initiators:

The process according to the invention is a free-radical polymerization. The way in which this polymerization is initiated is not critical; in this respect, initiation by means of peroxidic initiators, azo initiators or redox systems, or by a photochemical route, is possible. Among these initiators, the azo initiators are preferred.

Azo initiators used may, for example, be the following compounds:
2,2′-azobis(isobutyronitrile), 2,2′-azobis(2-cyano-2-butane), dimethyl 2,2′-azobis(dimethyl isobutyrate), 4,4′-azobis(4-cyanopentanoic acid), 2-(t-butylazo)-2-cyanopropane, 2,2′-azobis[2-methyl-N-(1,1)-bis(hydroxymethyl)-2-hydroxyethyl]propionamide, 2,2′-azobis[2-methyl-N-hydroxyethyl]propionamide, 2,2′-azobis(N,N-dimethyleneisobutyramidine)dihydrochloride, 2,2′-azobis(2-amidinopropane) dihydrochloride, 2,2′-azobis(N,N′-dimethyleneisobutyramine), 2,2′-azobis(2-methyl-N-[1,1-bis(hydroxymethyl)-2-hydroxyethyl]propionamide), 2,2′-azobis(2-methyl-N-[1,1-bis(hydroxymethypethyl]propionamide), 2,2′-azobis[2-methyl-N-(2-hydroxyethyl)-propionamide], 2,2′-azobis(isobutyramide) dihydrate, 2,2′-azobis(2,2,4-trimethylpentane), 2,2′-azobis(2-methylpropane), 1,1′-azobis(cyclohexane-1-carbonitrile), 2,2′-azobis[N-(2-propenyl)-2-methylpropionamide], 1-[(1-cyano-1-methylethyl)azo]formamide, 2,2′-azobis(N-butyl-2-methylpropionamide), 2,2′-azobis-(N-cyclohexyl-2-methylpropionamide) and 2,2′-azobis(2,4,4-trimethylpentane).

Typically, the azo initiators are used in an amount of 10⁻⁴ to 10⁻¹ mol/l, preferably in an amount of 10⁻⁴ to 10⁻² mol/l. By matching the ratio of the amount of initiator used to the amount of the chain transfer agent used, it is possible to influence both the reaction kinetics and the molecular structure (molecular weight, polydispersity) in a controlled manner.

Peroxidic initiators used may, for example, be the following peroxo compounds having an —O—O— unit: hydrogen peroxide, peroxodisulphates, peroxodiphosphates, hydroperoxides, peracids, peresters, peranhydrides and peroxides having two organic radicals. Salts of peroxodisulphuric acid and peroxodiphosphoric acid used may be sodium, potassium and ammonium salts. Suitable hydroperoxides are, for example, t-butyl hydroperoxide, cumene hydroperoxide, pinane hydroperoxide and p-menthane hydroperoxide. Suitable peroxides having two organic radicals are dibenzoyl peroxide, 2,4-dichlorobenzoyl peroxide, 2,5-dimethylhexane 2,5-di-t-butylperoxide, bis(t-butylperoxyisopropyl)benzene, t-butyl cumyl peroxide, di-t-butyl peroxide, dicumyl peroxide, t-butyl perbenzoate, t-butyl peracetate, 2,5-dimethylhexane 2,5-diperbenzoate, t-butyl per-3,5,5-trimethylhexanoate. Preference is given to using p-menthane hydroperoxide, cumene hydroperoxide, pinane hydroperoxide or 1,1-di(tert-butylperoxy)-3,3,5-trimethylcyclohexane.

In an alternative embodiment, azo initiators or peroxidic initiators having a prolonged breakdown time are used. It has been found here to be useful to choose the azo initiator or the peroxidic initiator such that the half-life of the respective initiator in the chosen solvent at a temperature of 70° C. to 200° C., preferably 80° C. to 175° C., more preferably 85° C. to 160° C. and especially 90° C. to 150° C. is 10 hours or more than 10 hours. Preference is given to azo initiators which, at a temperature of 70° C. to 200° C., preferably 80° C. to 175° C., more preferably 85° C. to 160° C. and most preferably 90″C to 150° C., have a half-life of 10 hours or more than 10 hours in the chosen solvent. Particular preference is given to using azo initiators of the following structural formulae (Ini-1)-(Ini-6):

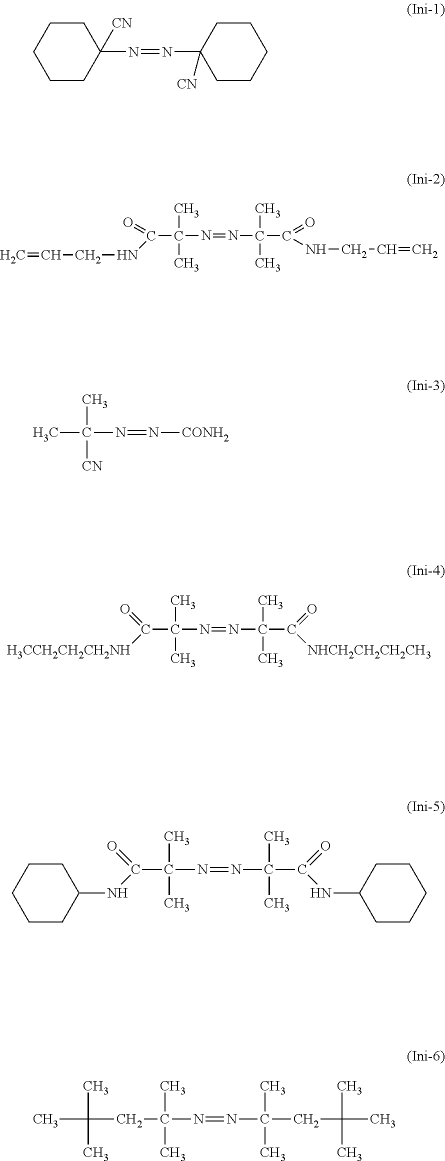

Very particular preference is given to the use of the initiators of the formula (Ini 1), (Ini-2) and (Ini-3). The aforementioned azo initiators of the structural formulae (Ini-1)-(Ini-6) are commercially available, for example from Wako Pure Chemical Industries, Ltd.

The term “half-life” is familiar to those skilled in the art in connection with initiators. Merely by way of example: a half-life of 10 hours in a solvent at a particular temperature means, specifically, that half of the initiator has broken down after 10 hours under these conditions.

When the aforementioned preferred initiators having a relatively high breakdown temperature are used, especially the azo initiators mentioned, it is possible to synthesize nitrile rubbers having comparatively high mean molecular weights Mw (weight-average molecular weight) and Mn (number-average molecular weight), which simultaneously also feature high linearity. This is expressed by correspondingly low Mooney relaxation values, measured by ISO 289 Parts 1 & 2 or alternatively to ASTM D1646.

Redox systems used may be the systems which follow, composed of an oxidizing agent and a reducing agent. The choice of suitable amounts of oxidizing agent and reducing agent is sufficiently familiar to the person skilled in the art.

When redox systems are used, salts of transition metal compounds such as iron, cobalt or nickel are frequently used additionally, in combination with suitable complexing agents such as sodium ethylenediaminetetraacetate, sodium nitrilotriacetate and trisodium phosphate or tetrapotassium diphosphate.

Oxidizing agents used may be, for example, any peroxo compounds which have been mentioned previously for the peroxidic initiators.

Reducing agents used in the process according to the invention may, for example, be as follows: sodium formaldehydesulphoxylate, sodium benzaldehydesulphoxylate, reducing sugars, ascorbic acid, sulphenates, sulphinates, sulphoxylates, dithionite, sulphite, metabisulphite, disulphite, sugars, urea, thiourea, xanthogenates, thioxanthogenates, hydrazinium salts, amines and amine derivatives such as aniline, dimethylaniline, monoethanolamine, diethanolamine or triethanolamine. Preference is given to using sodium formaldehydesulphoxylate.

The free-radical polymerization can also be initiated photochemically as described hereinafter: for this purpose, a photoinitiator which is excited by irradiation by means of light of suitable wavelength and initiates a free-radical polymerization is added to the reaction mixture. It should be noted here that, for the optimal initiation of free-radical polymerization, the irradiation time is dependent on the power of the emitter, on the distance between the emitter and the reaction vessel and on the irradiation area. However, it is possible for the person skilled in the art to discover the optimal irradiation time in a straightforward manner by various test series. The choice of the suitable amount of initiator is also possible for the person skilled in the art without difficulties and serves to influence the time/conversion characteristics of the polymerization.

Photochemical initiators used may, for example, be the following: benzophenone, 2-methylbenzophenone, 3,4-dimethylbenzophenone, 3-methylbenzophenone, 4,4′-bis(diethylamino)benzophenone, 4,4′-dihydroxybenzophenone, 4,4′-bis[2-(1-propenyl)-phenoxy]benzophenone, 4-(diethylamino)benzophetione, 4-(dimethylamino)benzophenone, 4-benzoylbiphenyl, 4-hydroxybenzophenone, 4-methylbenzophenone, benzophenone-3,3′,4,4′-tetracarboxylic dianhydride, 4,4′-bis(dimethylamino)benzophenone, acetophenone, 1-hydroxycyclohexyl phenyl ketone, 2,2-diethoxyacetophenone, 2,2-dimethoxy-2-phenyl-acetophenone, 2-benzyl-2-(dimethylamino)-4′-morpholinobutyrophenone, 2-hydroxy-2-methyl-propiophenone, 2-hydroxy-4′-(2-hydroxyethoxy)-2-methylpropiophenone, 3′-hydroxyaceto-phenone, 4′-ethoxyacetophenone, 4′-hydroxyacetophenone, 4′-phenoxyacetophenone, 4′-tert-butyl-2′,6′-dimethylacetophenone, 2-methyl-4′-(methylthio)-2-morpholinopropiophenone, diphenyl-(2,4,6-trimethylbenzoyl)phosphine oxide, phenylbis(2,4,6-trimethylbenzoyl)phosphine oxide, methylbenzoyl formate, benzoin, 4,4′-dimethoxybenzoin, benzoin methyl ether, benzoin ethyl ether, benzoin isopropyl ether, benzoin isobutyl ether, 4,4′-dimethylbenzil, hexachloro-cyclopentadiene or combinations thereof.

### Solvents

The process according to the invention is preferably conducted in an organic solvent or diluent. This applies both to the polymerization step and to the actual coupling reaction. Large amounts of water, as in the case of emulsion polymerization, are thus not present in the reaction system. Smaller amounts of water in the order of magnitude of up to 5% by weight, preferably to 1% by weight (based on the amount of the organic solvent), may quite possibly be present in the reaction system. What is crucial is that the amount of water present must be kept sufficiently low that there is no precipitation of the NBR polymer which forms. It should be made clear at this point that the process according to the invention is not an emulsion polymerization.

Examples of suitable organic solvents include acetone, acetonitrile, dimethylacetamide, monochlorobenzene, dichloromethane, toluene, ethyl acetate, 1,4-dioxane, t-butanol, isobutyronitrile, 3-propanone, dimethyl carbonate, 4-methylbutan-2-one and methyl ethyl ketone. Preference is given to polar solvents having a Hildebrand dissolution parameter δ (δ=((ΔH_{V}−RT)Vₘ)^{1/2}[(MPa)^{1/2}]) (Vₘ=molar volume; ΔH_{V}=enthalpy of vaporization; R=ideal gas constant)) within a range between 15.5 and 26 (MPa)^{1/2}.

The crucial factor for the suitability of a solvent is that the nitrile rubber produced remains completely in solution during the polymerization, during the subsequent workup and during the coupling step. It is not possible to use solvents which intervene in the reaction as transfer reagents, for example carbon tetrachloride, thiols and further solvents of this kind which are known as such to those skilled in the art, and also solvents having strong UV-absorbing action.

It is likewise possible to use a mixture of two or more organic solvents. It is also possible to use solvents which satisfy the above requirements and have a boiling point below that of acrylonitrile, for example methyl tert-butyl ether (MTBE).

### Temperature:

The polymerization process according to the invention is typically conducted at a temperature within a range from 60° C. to 150° C., preferably within a range from 70° C. to 130° C., more preferably within a range from 80° C. to 120° C. and especially within a range from 90° C. to 110° C. If the temperature selected is even lower, the polymerization is slowed correspondingly. At considerably higher temperatures, it is not impossible that the initiator used will break down too quickly or that the RAFT agent will decompose. Especially when peroxidic initiators are used, it is not impossible that the chain transfer agent will be oxidized under some circumstances.

The coupling process according to the invention can generally be effected within broad concentration ranges and at any suitable temperature. Preferably, the coupling is conducted at a temperature in the range from 0 to 200° C., more preferably 20 to 140° C., especially about room temperature (22-26″C). The coupling can generally be effected under air.

Reaction:

In the case of initiation by peroxo compounds or azo initiators, the process according to the invention is typically conducted in such a way that the α,β-unsaturated nitrile and the further copolymerizable monomers optionally used, the solvent, the initiator and the chain transfer agent(s) are initially charged in a reaction vessel and then the conjugated diene(s) is/are metered in. The polymerization is subsequently started by increasing the temperature.

In the case of initiation by means of a redox system, the oxidizing agent is typically metered into the reaction vessel together with one of the monomers. The polymerization is subsequently started by adding the reducing agent.

In order to obtain specific ratios of the respective monomers in the co-/terpolymer, it is advisable, and entirely familiar to the person skilled in the art, to undertake corresponding modifications in terms of the metered addition (for example by further metered addition of the respective monomer, of amounts of initiator, amounts of chain transfer agent or solvent). These further metered additions may either be continuous or else batchwise in individual portions. The further metered addition of the monomers or else the further metered addition of initiator can be effected either continuously or else in individual portions.

To establish a suitable molecular weight, and for the purpose of attaining the desired conversion, it has been found to be useful in one embodiment of the process according to the invention to meter in further initiator once or more than once in the course of the polymerization reaction.

The conjugated diene in the nitrile rubber may be any conjugated diene. Preference is given to using (C₄-C₆) conjugated dienes. Particular preference is given to 1,2-butadiene, 1,3-butadiene, isoprene, 2,3-dimethylbutadiene, piperylene or mixtures thereof. Especially preferred are 1,3-butadiene and isoprene or mixtures thereof. Very particular preference is given to 1,3-butadiene. α,β-Unsaturated nitriles used may be any known α,β-unsaturated nitrites, preference being given to (C₃-C₅)-α,β-unsaturated nitrites such as acrylonitrile, methacrylonitrile, ethacrylonitrile or mixtures thereof. Particular preference is given to acrylonitrile.

A particularly preferred nitrile rubber is a copolymer of acrylonitrile and 1,3-butadiene.

Further copolymerizable termonomers used may be aromatic vinyl monomers, preferably styrene, α-methylstyrene and vinylpyridine, fluorinated vinyl monomers, preferably fluoroethyl vinyl ether, tluoropropyl vinyl ether, o-fluoromethylstyrene, vinyl pentafluorobenzoate, difluoroethylene and tetrafluoroethylene, or else copolymerizable antiageing monomers, preferably N-(4-anilinophenyl)acrylamide; N-(4-anilinophenyl)methacrylamide, N-(4-anilinophenyl)-cinnamide, N-(4-anilinophenyl)crotonamide, N-phenyl-4-(3-vinylbenzyloxy)aniline and N-phenyl-4-(4-vinylbenzyloxy)aniline, and also non-conjugated dienes such as 4-cyanocyclohexene and 4-vinylcyclohexene, or else alkynes such as 1- or 2-butyne.

Alternatively, further copolymerizable termonomers used may be copolymerizable termonomers containing carboxyl groups, for example α,β-unsaturated monocarboxylic acids, esters thereof, α,β-unsaturated dicarboxylic acids, the mono- or diesters thereof, or the corresponding anhydrides or amides thereof. α,β-Unsaturated monocarboxylic acids used may preferably be acrylic acid and methacrylic acid.

It is also possible to use esters of the α,β-unsaturated monocarboxylic acids, preferably the alkyl esters and alkoxyalkyl esters thereof. Preference is given to the alkyl esters, especially C₁-C₁₈ alkyl esters of the α,β-unsaturated monocarboxylic acids. Particular preference is given to alkyl esters, especially C₁-C₁₈ alkyl esters, of acrylic acid or of methacrylic acid, especially methyl acrylate, ethyl acrylate, propyl acrylate, n-butyl acrylate, tert-butyl acrylate, 2-ethylhexyl acrylate, n-dodecyl acrylate, methyl methacrylate, ethyl methacrylate, butyl methacrylate and 2-ethylhexyl methacrylate. Preference is also given to alkoxyalkyl esters of the α,β-unsaturated monocarboxylic acids, more preferably alkoxyalkyl esters of acrylic acid or of methacrylic acid, especially C₂-C₁₂-alkoxyalkyl esters of acrylic acid or of methacrylic acid, most preferably methoxymethyl acrylate, methoxyethyl (meth)acrylate, ethoxyethyl (meth)acrylate and methoxymethyl (meth)acrylate. It is also possible to use mixtures of alkyl esters, for example those mentioned above, with alkoxyalkyl esters, for example in the form of those mentioned above. It is also possible to use cyanoalkyl acrylates and cyanoalkyl methacrylates in which the number of carbon atoms in the cyanoalkyl group is 2-12, preferably a-cyanoethyl acrylate, β-cyanoethyl acrylate and cyanobutyl methacrylate. It is also possible to use hydroxyalkyl acrylates and hydroxyalkyl methacrylates in which the number of carbon atoms in the hydroxyalkyl groups is 1-12, preferably 2-hydroxyethyl acrylate, 2-hydroxyethyl methacrylate and 3-hydroxypropyl acrylate; also usable are fluorine-substituted acrylates or methacrylates containing benzyl groups, preferably fluorobenzyl acrylates, and fluorobenzyl methacrylate. It is also possible to use acrylates and methacrylates containing fluoroalkyl groups, preferably trifluoroethyl acrylate and tetrafluoropropyl methacrylate. It is also possible to use α,β-unsaturated carboxylic esters containing amino groups, such as dimethylaminomethyl acrylate and diethylaminoethyl acrylate.

Further copolymerizable monomers used may also be α,β-unsaturated dicarboxylic acids, preferably maleic acid, fumaric acid, crotonic acid, itaconic acid, citraconic acid and mesaconic acid.

It is additionally possible to use α,β-unsaturated dicarboxylic anhydrides, preferably maleic anhydride, itaconic anhydride, citraconic anhydride and mesaconic anhydride. It is additionally possible to use mono- or diesters of α,β-unsaturated dicarboxylic acids.

These α,β-unsaturated dicarboxylic mono- or diesters may, for example, be alkyl, preferably C₁-C₁₀-alkyl, especially ethyl, n-propyl, isopropyl, n-butyl, tert-butyl, n-pentyl or n-hexyl, alkoxyalkyl, preferably C₂-C₁₂-alkoxyalkyl, more preferably C₃-C₈-alkoxyalkyl, hydroxyalkyl, preferably C₁-C₁₂-hydroxyalkyl, more preferably C₂-C₈-hydroxyalkyl, cycloalkyl, preferably C₅-C₁₂-cycloalkyl, more preferably C₆-C₁₂-cycloalkyl, alkylcycloalkyl, preferably C₆-C₁₂-alkylcycloalkyl, more preferably C₇-C₁₀-alkylcycloalkyl, aryl, preferably C₆-C₁₄-aryl, mono- or diesters, where the diesters may also be mixed esters in each case.

Particularly preferred alkyl esters of α,β-unsaturated monocarboxylic acids are methyl (meth)acrylate, ethyl (rneth)acrylate, propyl (meth)acrylate, n-butyl (meth)acrylate, t-butyl (meth)acrylate, hexyl (meth)acrylate, 2-ethylhexyl (meth)acrylate, octyl (meth)acrylate, 2-propylheptyl acrylate and lauryl (meth)acrylate. More particularly, n-butyl acrylate is used.

Particularly preferred alkoxyalkyl esters of the α,β-unsaturated monocarboxylic acids are methoxyethyl (meth)acrylate, ethoxyethyl (meth)acrylate and methoxymethyl (meth)acrylate. More particularly, methoxyethyl acrylate is used.

Particularly preferred hydroxyalkyl esters of the α,β-unsaturated monocarboxylic acids are hydroxyethyl (meth)acrylate, hydroxypropyl (meth)acrylate and hydroxybutyl (meth)acrylate.

Other esters of the α,β-unsaturated monocarboxylic acids used are, for example, polyethylene glycol (meth)acrylate, polypropylene glycol (meth)acrylate, glycidyl (meth)acrylate, epoxy(meth)acrylate, N-(2-hydroxyethypacrylamide, N-(2-hydroxymethyl)acrylamide and urethane (meth)acrylate.

Examples of α,β-unsaturated dicarboxylic monoesters include
monoalkyl maleates, preferably monomethyl maleate, monoethyl maleate, monopropyl maleate and mono-n-butyl maleate;
monocycloalkyl maleates, preferably monocyclopentyl maleate, monocyclohexyl maleate and monocycloheptyl maleate;
monoalkylcycloalkyl maleates, preferably monomethylcyclopentyl maleate and monoethylcyclohexyl maleate;
monoaryl maleates, preferably monophenyl maleate;
monobenzyl maleates, preferably monobenzyl maleate;
monoalkyl fumarates, preferably monomethyl fumarate, monoethyl fumarate, monopropyl fumarate and mono-n-butyl fumarate;
monocycloalkyl fumarates, preferably monocyclopentyl fumarate, monocyclohexyl fumarate and monocycloheptyl fumarate;
monoalkylcycloalkyl fumarates, preferably monomethylcyclopentyl fumarate and monoethylcyclohexyl fumarate;
monoaryl fumarates, preferably monophenyl fumarate;
monobenzyl fumarates, preferably monobenzyl fumarate;
monoalkyl citraconates, preferably monotnethyl citraconate, monoethyl citraconate, monopropyl citraconate and mono-n-butyl citraconate;
monocycloalkyl citraconates, preferably monocyclopentyl citraconate, monocyclohexyl citraconate and monocycloheptyl citraconate;
monoalkylcycloalkyl citraconates, preferably monomethylcyclopentyl citraconate and monoethylcyclohexyl citraconate;
monoaryl citraconates, preferably monophenyl citraconate;
monobenzyl citraconates, preferably monobenzyl citraconate;
monoalkyl itaconates, preferably monomethyl itaconate, monoethyl itaconate, monopropyl itaconate and mono-n-butyl itaconate;
monocycloalkyl itaconates, preferably monocyclopentyl itaconate, monocyclohexyl itaconate and monocycloheptyl itaconate;
monoalkylcycloalkyl itaconates, preferably monomethylcyclopentyl itaconate and monoethylcyclohexyl itaconate;
monoaryl itaconates, preferably monophenyl itaconate;
monobenzyl itaconates, preferably monobenzyl itaconate;
monoalkyl mesaconates, preferably monoethyl mesaconate.

α,β-Unsaturated dicarboxylic diesters used may be the analogous diesters based on the aforementioned monoester groups, where the ester groups may also be chemically different groups.

It is additionally possible to use, as further copolymerizable monomers, free-radically polymerizable compounds containing two or more olefinic double bonds per molecule. Examples of such di- or polyunsaturated compounds are di- or polyunsaturated acrylates, methacrylates or itaconates of polyols, for example 1,6-hexanediol diacrylate (HDODA), 1,6-hexanediol dimethacrylate, ethylene glycol diacrylate, ethylene glycol dimethacrylate (EGDMA), diethylene glycol dimethacrylate, triethylene glycol diacrylate, butane-1,4-diol diacrylate, propane-1,2-diol diacrylate, butane-1,3-diol dimethacrylate, neopentyl glycol diacrylate, trimethylolpropane diacrylate, trimethylolpropane dimethacrylate, trimethylolethane diacrylate, trimethylolethane dimethacrylate, trimethylolpropane triacrylate, trimethylolpropane trimethacrylate (TMPTMA), glyceryl di- and triacrylate, pentaerythrityl di-, tri- and tetraacrylate or -methacrylate, dipenterythrityl tetra-, penta- and hexaacrylate or -methacrylate or -itaconate, sorbitol tetraacrylate, sorbitol hexamethacrylate, diacrylates or dimethacrylates of 1,4-cyclohexanediol, 1,4-dimethyl-olcyclohexane, 2,2-bis(4-hydroxyphenyl)propane, of polyethylene glycols or of oligoesters or oligourethanes having terminal hydroxyl groups. Polyunsaturated monomers used may also be to acrylamides, for example methylenebisacrylamide, hexamethylene-1,6-bisacrylamide, diethylene-triaminetrismethacrylamide, bis(methacrylamidopropoxy)ethane or 2-acrylamidoethyl acrylate. Examples of polyunsaturated vinyl and allyl compounds are divinylbenzene, ethylene glycol divinyl ether, diallyl phthalate, allyl methacrylate, diallyl maleate, triallyl isocyanurate or triallyl phosphate.

When termonomers of this kind are used, it is advantageously possible to conduct the polymerization up to high conversions and at the same time to produce nitrite rubbers which have a comparatively high mean molecular weight Mw (weight average) and/or Mn (number average) hut are nevertheless gel-free.

The proportions of conjugated diene and α,β-unsaturated nitrile in the NBR polymers obtained may vary within wide ranges. The proportion of, or of the sum total of, the conjugated dienes is typically in the range from 40 to 90% by weight, preferably in the range from 50 to 85% by weight, based on the overall polymer. The proportion of, or of the sum total of, the 0.8-unsaturated nitriles is typically 10 to 60% by weight, preferably 15 to 50% by weight, based on the overall polymer. The proportions of the monomers add up to 100% by weight in each case. According to the nature of the termonomer(s), the additional monomers may be present in amounts of 0 to 40% by weight, based on the overall polymer. In this case, corresponding proportions of the conjugated diene(s) and/or of the α,β-unsaturated nitrile(s) are replaced by the proportions of the additional monomers, where the proportions of all the monomers add up to 100% by weight in each case.

If the termonomers are those monomers which form tertiary radicals (e.g. methacrylic acid), it has been found to be useful to use these in amounts of 0 to 10% by weight.

It should be noted that the aforementioned restriction of the additional monomers to max. 40% applies only to the situation where the total amount of monomers is metered into the polymerization mixture at the start or during the reaction (i.e. for production of random terpolymer systems). Of course, it is possible to use an optionally hydrogenated nitrite rubber produced in accordance with the invention as a macro-chain transfer agent because of the fact that it has fragments of the chain transfer agent(s) used in the polymer backbone and/or the end groups, and to use it in any amount, for example, for generation of block systems through reaction with suitable monomers.

The glass transition temperatures of the optionally hydrogenated nitrite rubbers of the invention are in the range from −70° C. to +20° C., preferably in the range of −60° C. to 10° C.

Because of the living character of the polymerization by means of the process according to the invention, it is possible to obtain nitrite rubbers with a narrow molecular weight distribution. It is possible to prepare nitrite rubbers with a polydispersity index in the range from 1.0 to 2.9, preferably in the range from 1.1 to 2.8, more preferably in the range from 1.15 to 2.7 and especially in the range from 1.2 to 2.6.

Because of the living character of the polymerization by means of the process according to the invention, it is even possible to obtain nitrile rubbers with an extremely narrow molecular weight distribution. It is possible to prepare nitrite rubbers with a polydispersity index in the range from 1.1 to 2.5, preferably within a range from 1.3 to 2.4, more preferably within a range from 1.4 to 2.2, especially within a range from 1.5 to 2.0, most preferably within a range from 1.3 to less than 2.

The process according to the invention allows, through control of the chain transfer agent concentration, the exact setting of the desired molecular weight, and additionally, through use of the chain transfer agents, also the formation of selected polymer architectures (for example production of blocks, grafts onto polymer backbones, surface attachment, the use of termonomers having more than one C═C double bond, and further polymer modifications known to those skilled in the art), and also controlled molecular weight distributions from extremely narrow up to broad distributions, from monomodal through bimodal to multimodal distributions. The nitrite rubbers formed in a controlled manner by means of these methods may have a polydispersity index PDI=Mw/Mn where Mw is the weight-average and Mn the number-average molecular weight, in the range from 1.1 to 8.0, preferably in the range from 1.15 to 7.0, more preferably in the range from 1.2 to 6.0 and especially in the range from 1.3 to 5.0.

### Hydrogenation:

The present invention further provides hydrogenated nitrile rubbers, whereby the hydrogenation c) directly follows the first polymerization step a) when the chain transfer agent of the general formula (VI) is used, i.e. prior to attachment of the tetrazole group, and b) when the chain transfer agent of the general formula (I) is used, i.e. including the tetrazole group, with no need for any prior isolation of the nitrile rubber. The hydrogenation can be conducted immediately after the polymerization, if desired even in the same reactor. This leads to a substantial simplification and hence to economic advantages in the production of the HNBR.

The hydrogenation can be conducted using homogeneous or heterogeneous hydrogenation catalysts as described in WO 2011/032832.

In the case of the nitrile rubbers to be coupled which are obtained using the chain transfer agents of the general formula (I), a hydrogenation is not conducted until after the coupling. The corresponding hydrogenation can likewise be conducted as specified above.

It is a feature both of the inventive coupled nitrile rubbers and of the hydrogenated coupled nitrile rubbers that, compared to the optionally hydrogenated nitrile rubbers in which the nitrile rubber is obtained by emulsion polymerization, they are completely free of emulsifier and also do not contain any salts as typically used for coagulation of the latices after the emulsion polymerization for the purpose of precipitation of the nitrile rubber.

The present invention further provides vulcanizable mixtures comprising the coupled, optionally hydrogenated nitrile rubber and at least one crosslinker. In a preferred embodiment, the vulcanizable mixtures additionally comprise at least one filler.

Optionally, vulcanizable mixtures of this kind may also comprise one or more additives familiar to the person skilled in the art for rubbers. These include ageing stabilizers, reversion stabilizers, light stabilizers, ozone stabilizers, processing aids, plasticizers, mineral oils, tackifiers, blowing agents, dyes, pigments, waxes, resins, extenders, organic acids, vulcanization retardants, metal oxides, and further filler activators, for example triethanolamine, trimethylolpropane, polyethylene glycol, hexanetriol, aliphatic trialkoxysilanes, or other additives known in the rubber industry (Ullmann's Encyclopedia of Industrial Chemistry, VCH Verlagsgesellschaft mbH, D-69451 Weinheim, 1993, vol A 23 “Chemicals and Additives”, p. 366-417).

Examples of useful crosslinkers include peroxidic crosslinkers such as bis(2,4-dichlorobenzyl) peroxide, dibenzoyl peroxide, bis(4-chlorobenzoyl) peroxide, 1,1-bis-(t-butylperoxy)-3,3,5-trimethylcyclohexane, tert-butyl perbenzoate, 2,2-bis(t-butylperoxy)butene, 4,4-di-tert-butyl peroxynonylvalerate, dicumyl peroxide, 2,5-dimethyl-2,5-di(t-butylperoxy)hexane, tert-butyl cumyl peroxide, 1,3-bis(t-butylperoxyisopropyl)benzene, di-t-butyl peroxide and 2,5-dimethyl-2,5-di(t-butylperoxy)hex-3-yne.

It may be advantageous to use, as well as these peroxidic crosslinkers, also further additives which can help to increase the crosslinking yield: suitable examples for this purpose are triallyl isocyanurate, triallyl cyanurate, trimethylolpropane tri(meth)acrylate, triallyl trimellitate, ethylene glycol dimethacrylate, butanediol dimethacrylate, trimethylolpropane trimethacrylate, zinc acrylate, zinc diacrylate, zinc methacrylate, zinc dimethacrylate, 1,2-polybutadiene or N,N′-m-phenylene-dimaleimide.

The total amount of the crosslinker(s) is typically in the range from 1 to 20 phr, preferably in the range from 1.5 to 15 phr and more preferably in the range from 2 to 10 phr, based on the optionally hydrogenated nitrile rubber.

The crosslinkers used may also be sulphur in elemental soluble or insoluble form, or sulphur donors.

Examples of useful sulphur donors include dimorpholyl disulphide (DTDM), 2-morpholino-dithiobenzothiazole (MBSS), caprolactam disulphide, dipentamethylenethiuram tetrasulphide (DPTT) and tetramethylthiuram disulphide (TMTD).

In the case of sulphur vulcanization of the inventive nitrile rubbers too, it is possible to use further additions which can help to increase the crosslinking yield. In principle, the crosslinking can also be effected with sulphur or sulphur donors alone. Conversely, the crosslinking of the inventive, optionally hydrogenated nitrile rubber can also be effected only in the presence of the abovementioned additions, i.e. without addition of elemental sulphur or sulphur donors.

Suitable additions which can help to increase the crosslinking yield are, for example, dithiocarbamates, thiurams, thiazoles, sulphenamides, xanthogenates, guanidine derivatives, caprolactams and thiourea derivatives.

Dithiocarbamates used may be, for example: ammonium dimethyldithiocarbamate, sodium diethyldithiocarbamate (SDEC), sodium dibutyldithiocarbamate (SDBC), zinc dimethyldithiocarbamate (ZDMC), zinc diethyldithiocarbamate (ZDEC), zinc dibutyldithio-carbamate (ZDBC), zinc ethylphenyldithiocarbamate (ZEPC), zinc dihenzyldithiocarbamate (ZBEC), zinc pentamethylenedithiocarhamate (Z5MC), tellurium diethyldithiocarbamate, nickel dibutyldithiocarbamate, nickel dimethyldithiocarbamate and zinc diisononyldithiocarbamate.

Thiurams used may be, for example: tetramethylthiuram disulphide (TMTD), tetramethylthiuram monosulphide (TMTM), dimethyldiphenylthiuram disulphide, tetrabenzylthiuram disulphide, dipentatnethylenethiuratn tetrasulphide and tetraethylthiuram disulphide (TETD).

Thiazoles used may be, for example: 2-mercaptobenzothiazole (MBT), dibenzothiazyl disulphide (MBTS), zinc mercaptobenzothiazole (ZMBT) and copper 2-mercaptobenzothiazole. Sulphenamide derivatives used may be, for example: N-cyclohexyl-2-benzothiazylsulphenamide (CBS), N-tert-butyl-2-benzthiazylsulphenamide (TBBS), N,N′-dicyclohexyl-2-benzthiazyl-sulphenamide (DCBS),2-morpholinothiobenzothiazole (MBS), N-oxydiethylenethiocarbamyl-N-tert-butylsulphenamide and oxydiethylenethiocarbamyl-N-oxyethylenesulphen amide.

Xanthogenates used may be, for example: sodium dibutylxanthogenate, zinc isopropyldibutylxanthogenate and zinc dibutylxanthogenate.

Guanidine derivatives used may be, for example: diphenylguanidine (DPG), di-o-tolylguanidine (DOTG) and o-tolylbiguanide (OTBG).

Dithiophosphates used may be, for example: zinc dialkyldithiophosphates (chain length of the alkyl radicals C2 to C16), copper dialkyldithiophosphates (chain length of the alkyl radicals C₂ to C₁₆) and dithiophosphoryl polysulphide.

The caprolactam used may be, for example, dithiobiscaprolactam.

Thiourea derivatives used may be, for example, N,N′-diphenylthiourea (DPTU), diethylthiourea (DETU) and ethylenethiourea (ETU).

Likewise suitable as additions are, for example: zinc diaminediisocyanate, hexamethylenetetramine, 1,3-bis(citraconimidomethyl)benzene and cyclic disulphanes.

Said additions, and also the crosslinking agents, can be used either individually or in mixtures. Preferably, the following substances are used for the crosslinking of the nitrile rubbers: sulphur, 2-mercaptobenzthiazole, tetramethylthiuram disulphide, tetramethylthiuram monosulphide, zinc dibenzyldithiocarbamate, dipentamethylenethiuram tetrasulphide, zinc dialkyldithiophosphate, dimorpholyl disulphide, tellurium diethyldithiocarbamate, nickel dibutyldithiocarbamate, zinc dibutyldithiocarbamate, zinc dimethyldithiocarbamate and dithiobiscaprolactam.

The crosslinking agents and aforementioned additions can each be used in amounts of about 0.05 to 10 phr, preferably 0.1 to 8 phr, especially 0.5 to 5 phr (individual dosage, based in each case on the active substance), based on the optionally hydrogenated nitrile rubber.

In the inventive sulphur crosslinking, it may also be advisable, in addition to the crosslinking agents and abovementioned additions, also to use further inorganic or organic substances, for example: zinc oxide, zinc carbonate, lead oxide, magnesium oxide, calcium oxide, saturated or unsaturated organic fatty acids and zinc salts thereof, polyalcohols, amino alcohols, for example triethanolamine, and amines, for example dibutylamine, dicyclohexylamine, cyclohexylethylamine and polyether amines.

If the inventive coupled, optionally hydrogenated nitrile rubbers are those which include repeat units of one or more termonomers containing carboxyl groups, crosslinking can also be effected via the use of a polyamine crosslinker, preferably in the presence of a crosslinking accelerator. The polyamine crosslinker is not restricted, provided that it is (1) a compound containing either two or more amino groups (optionally also in salt form) or (2) a species which, during the crosslinking reaction, forms a compound containing two or more amino groups in situ. Preference is given to using an aliphatic or aromatic hydrocarbon compound in which at least two hydrogen atoms are replaced either by amino groups or else by hydrazide structures (the latter being a structure “—C(═O)NHNH₂”).

Examples of such polyamine crosslinkers (ii) are:
aliphatic polyamines, preferably hexamethylenediamine, hexamethylenediamine carbamate, tetramethylenepentamine, hexamethylenediamine-cinnamaldehyde adduct or hexamethylenediamine dibenzoate;
aromatic polyamines, preferably 2,2-bis(4-(4-aminophenoxy)phenyl)propane, 4,4′-methylenedianiline, m-phenylenediamine, p-phenylenediamine or 4,4′-methylenebis(o-chloroaniline);

compounds having at least two hydrazide structures, preferably isophthalic dihydrazide, adipic dihydrazide or sebacic dihydrazide.

Particular preference is given to hexamethylenediamine and hexamethylenediamine carbamate.

The amount of the polyamine crosslinker in the vulcanizable mixture is typically in the range from 0.2 to 20 parts by weight, preferably in the range from 1 to 15 parts by weight and more preferably in the range from 1.5 to 10 parts by weight, based on 100 parts by weight of the optionally hydrogenated nitrile rubber.

Crosslinking accelerators used in combination with the polyamine crosslinker may be any of those known to those skilled in the art, preferably a basic crosslinking accelerator. It is possible to use, for example, tetramethylguanidine, tetraethylguanidine, diphenylguanidine, di-o-tolylguanidine (DOTG), o-tolylbiguanidine and di-o-tolylguanidine salt of dicatecholboric acid. It is additionally possible to use aldehyde-amine crosslinking accelerators, for example n-butylaldehyde-aniline. Particular preference is given to using, as the crosslinking accelerator, at least one bi- or polycyclic aminic base. These are known to those skilled in the art. The following are especially suitable: 1,8-diazabicyclo[5.4.0]undec-7-ene (DBD), 1,5-diazabicyclo[4.3.0]-5-nonene (DBN), 1,4-diazabicyclo[2.2.2]octane (DABCO), 1,5,7-triazabicyclo[4.4.0]dec-5-ene (TBD), 7-methyl-1,5,7-triazabicyclo[4.4.0]dec-5-ene (MTBD).

The amount of the crosslinking accelerator in this case is typically within a range from 0.5 to 10 parts by weight, preferably 1 to 7.5 parts by weight, especially 2 to 5 parts by weight, based on 100 parts by weight of the optionally hydrogenated nitrile rubber.

The vulcanizable mixture based on the inventive optionally hydrogenated nitrile rubber may in principle also comprise scorch retardants. These include cyclohexylthiophthalimide (CTP). N,N′-dinitrosopentamethylenetetramine (DNPT), phthalic anhydride (PTA) and diphenylnitrosamine. Preference is given to cyclohexylthioplithalimide (CTP).

As well as the addition of the crosslinker(s), the inventive, optionally hydrogenated nitrile rubber can also be mixed with further customary rubber additives.

Fillers used may, for example, be carbon black, silica, barium sulphate, titanium dioxide, zinc oxide, calcium oxide, calcium carbonate, magnesium oxide, aluminium oxide, iron oxide, aluminium hydroxide, magnesium hydroxide, aluminium silicates, diatomaceous earth, talc, kaolins, bentonites, carbon nanotubes, Teflon (the latter preferably in powder form), or silicates.

Useful filler activators include organic silanes in particular, for example vinyltritnethyloxysilane, vinyldimethoxymethylsilane, vinyltriethoxysilane, vinyltris(2-methoxyethoxy)silane, N-cyclohexyl-3-aminopropyltrimethoxysilane, 3-aminopropyltrimethoxysilane, methyltrimethoxysilane, methyltriethoxysilane, dimethyldimethoxysilane, dimethyldiethoxysilane, trimethylethoxysilane, isooctyltrimethoxysilane, isooctyltriethoxysilane, hexadecyltrimethoxysilane or (octadecyl)methyldimethoxysilane. Further filler activators are, for example, interface-active substances such as triethanolamine and ethylene glycols with molecular weights of 74 to 10 000 g/mol. The amount of filler activators is typically 0 to 10 phr, based on 100 phr of the optionally hydrogenated nitrile rubber.

Ageing stabilizers added to the vulcanizable mixtures may be ageing stabilizers known from the literature. They are used typically in amounts of about 0 to 5 phr, preferably 0.5 to 3 phr, based on 100 phr of the optionally hydrogenated nitrile rubber.

Suitable phenolic ageing stabilizers are alkylated phenols, styrenized phenol, sterically hindered phenols such as 2,6-di-tert-butylphenol, 2,6-di-tert-butyl-p-cresol (BHT), 2,6-di-tert-butyl-4-ethylphenol, sterically hindered phenols containing ester groups, thioether-containing sterically hindered phenols, 2,2′-methylenebis(4-methyl-6-tert-butylphenol) (BPH) and sterically hindered thiobisphenols.

If discolouration of the nitrile rubber is unimportant, aminic ageing stabilizers are also used, for example mixtures of diaryl-p-phenylenediamines (DTPD), octylated diphenylamine (ODPA), phenyl-a-naphthylamine (PAN), phenyl-β-naphthylamine (PBN), preferably those based on phenylenediamine. Examples of phenylenediamines are N-isopropyl-N′-phenyl-p-phenylenediamine, N-1,3-dimethylbutyl-N′-phenyl-p-phenylenediamine (6PPD), N-1,4-dimethylpentyl-N′-phenyl-p-phenylenediamine (7PPD) and N,N′-bis-1,4-(1,4-dimethylpentyl)-p-phenylenediamine (77PD).

The other ageing stabilizers include phosphites such as tris(nonylphenyl) phosphite, polymerized 2,24-trimethyl-1,2-dihydroquinoline (TMQ), 2-mercaptobenzimidazole (MBI), methyl-2-mercaptobenzimidazole (MMBI), zinc methylmercaptobenzimidazole (ZMMBI). The phosphites are generally used in combination with phenolic ageing stabilizers. TMQ, MBI and MMBI are used particularly when vulcanization is effected using peroxides.

Examples of useful mould release agents include: saturated and partly unsaturated fatty acids and oleic acids and derivatives thereof (fatty acid esters, fatty acid salts, fatty alcohols, fatty acid amides), which are preferably used as a mixture constituent, and also products applicable to the mould surface, for example products based on low molecular weight silicone compounds, products based on fluoropolymers and products based on phenol resins.

The mould release agents are used as a mixture constituent in amounts of about 0 to 10 phr, preferably 0.5 to 5 phr, based on 100 phr of the optionally hydrogenated nitrile rubber.

Another option is reinforcement with strengthening agents (fibres) made of glass, according to the teaching of U.S. Pat. No. 4,826,721, and another is reinforcement by cords, woven fabrics, fibres made of aliphatic and aromatic polyamides (Nylon®, Aramid®), polyesters and natural fibre products.

The present invention further provides a process for producing vuicanizates, characterized in that the aforementioned vulcanizable mixture is subjected to crosslinking. The crosslinking is typically brought about either by means of at least one crosslinker or else by photochemical activation.

In the case of photochemically activated vulcanization, UV activators used may be those typically known to the person skilled in the art, for example benzophenone, 2-methylbenzophenone, 3,4-dimethylbenzophenone, 3-methylbenzophenone, 4,4′-bis(diethylamino)benzophenone, 4,4′-dihydroxybenzophenone, 4,4′-bis[2-(1-propenyl)phenoxy]benzophenone, 4-(diethylamino)-benzophenone, 4-(dimethy)amino)benzophenone, 4-benzoylbiphenyl, 4-hydroxybenzophenone, 4-methylbenzophenone, benzophenone-3,3′,4,4′-tetracarboxylic dianhydride, 4,4′-bis(dimethyl-amino)benzophenone, acetophenone, 1-hydroxy-cyclohexyl phenyl ketone, 2,2-diethoxyaceto-phenone, 2,2-dimethoxy-2-phenylacetophenone, 2-benzyl-2-(dimethylamino)-4′-morpholino-butyrophenone, 2-hydroxy-2-methylpropiophenone, 2-hydroxy-4′-(2-hydroxyethoxy)-2-methyl-propiophenone, 3′-hydroxyacetophenone, 4′-ethoxyacetophenone, 4′-hydroxyacetophenone, 4′-phenoxyacetophenone, 4′-tert-butyl-2′,6′-dimethylacetophenone, 2-methyl-4′-(methylthio)-2-morpholinopropiophenone, diphenyl(2,4,6-trimethylbenzoyl)phosphine oxide, phenylbis(2,4,6-trimethylbenzoyl)phosphine oxide, methylbenzoyl formate, benzoin, 4,4′-dimethoxybenzoin, benzoin methyl ether, benzoin ethyl ether, benzoin isopropyl ether, benzoin isobutyl ether, 4,4′-dimethylbenzil, hexachlorocyclopentadiene or combinations thereof.

### EXAMPLES

In the context of the examples which follow, it was possible by means of mass spectrometry (MS) to show clearly that the inventive nitrile rubber contains, as an end group of the polymer chains, a chain transfer agent fragment based on the chain transfer agent used.

The following abbreviations are used occasionally hereinafter:
ACN acrylonitrile
1,3-BD 1,3-butadiene
DoPAT dodecylpropanoic acid trithiocarbonate
tetrazole-DoPAT (etrazole-functionalized dodecylpropanoic acid trithiocarbonate
Vazo® 88: 1,1′-azobis(cyclohexanecarbonitrile) (DuPont)
M_{w} weight-average molecular weight
Mₙ number-average molecular weight
PDI polydispersity index (quotient of M_{w} and Mₙ)
DMF dimethylformamide

### Molecular Weights and Polydispersity Index:

The determination of the molecular weights in the form of the number-average molecular weight (Mₙ) and the weight-average molecular weight (M_{w}) and of the polydispersity index was effected by means of gel permeation chromatography (GPC) to DIN 55672-1 (Part 1: Tetrahydrofuran THF as solvent).

### Example 1

### Synthesis of the tetrazole-functional trithiocarbonate RAFT chain transfer agent 34(2-(((dodecylthio)carbonothioyl)thio)propanoyl)oxy)propyl-4-(2-phenyl-2H-tetrazol-5-yl)-benzoate (tetrazole-RAFT)

2-((Dodecylsulphanyl)carbonothioyl)sulphanylpropanoic acid (DoPAT, 5.000 g, 14.3 mmol), 1,3-propanediol (5.2 ml, 71.3 mmol) and 4-(dimethylamino)pyridine (0.346 g, 2.8 mmol) were dissolved in tetrahydrofuran (10 ml) and cooled to 0° C. with an ice/water bath. N,N′-Dicyclohexylearbodiimide (2.940 g, 2.8 mmol) was added, the cooling bath was removed and the mixture was stirred at room temperature overnight. The white precipitate was filtered off and discarded. The filtrate was concentrated under reduced pressure and then dissolved in diethyl ether (200 ml). The solution was extracted with 1M aqueous hydrochloric acid solution (4×200 ml) and then washed with saturated NaHCO₁ solution (200 ml). The organic phase was dried over MgSO₄ and concentrated under reduced pressure. By-products were removed by means of column chromatography using silica gel with hexane/ethyl acetate (3:1, v/v), and the 3-hydroxypropyl 2-(((dodecylthio)carbonothioyl)thio)propanoate (hydroxy-RAFT) intermediate was washed off the column with hexane/ethyl acetate (1:1 v/v, R_{f} 0.61) and dried under high vacuum. Yield: 4.367 g, 74%, yellow oil.

Hydroxy-RAFT (3.800 g, 9.3 mmol), 4-(dimethylamino)pyridine (0.026 g, 0.02 mmol) and 4-(2-phenyl-2H-tetrazol-5-yl)benzoic acid (2.890 g, 10.9 mmol) were dissolved in 20 ml of THF. The solution was cooled to 0° C. with an ice/water bath and N,N′-dicyclohexylcarbodiimide (2.450 g, 10.9 mmol) was added. The cooling bath was removed and the reaction was stirred at room temperature overnight. Volatile components were subsequently removed under reduced pressure and the residue was taken up in diethyl ether (200 ml). The organic phase was washed with 1M aqueous hydrochloric acid solution (4×200 ml) and then with saturated NaHCO₃ solution (200 ml), dried over MgSO₄ and concentrated under reduced pressure. The crude product was purified by chromatography on silica gel with an eluent mixture of hexane/ethyl acetate (3:1, v/v, R_{f} 0.47) and dried. Tetrazole-RAFT was obtained as a yellow solid. Yield: 2.760 g, 45%.

### Example 2

### Preparation of Tetrazole-Functional NBR in Organic Solution

The nitrile rubbers used in the following series of examples were prepared according to the base formulation specified for NBR 1 under the conditions specified in Table 1, with all feedstocks specified in parts by weight based on 100 parts by weight of the monomer mixture.

Before contact with 1,3-butadiene, all apparatus was freed of oxygen by evacuating and purging with nitrogen three times. In a typical polymerization, 44.3 mg of Vazo 88 (0.13 phm) and 953.0 mg of tetrazoleRAFT (2.7 phm) were dissolved in 18.8 ml of monochlorobenzene (59.1 phm), 16.4 ml of acrylonitrile (37.6 phm) were added and the mixture was degassed with nitrogen for 10 minutes. The monomer/initiator solution was transferred into the reactor, which was closed and freed of oxygen by evacuating/purging with nitrogen three times. 33.8 ml of 1,3-butadiene (62.4 phm) were metered in by a pressure burette and the reaction was commenced by heating to 100° C. The course of the polymerization was monitored by gravimetric determinations of conversion. After 5 h, the heat source was removed, excess 1,3-butadiene was removed by venting after the reactor had been cooled and the polymer was obtained by precipitation in ethanol. The polymer was subsequently dried under high vacuum.

TABLE 1
Preparation of tetrazole-functional NBRs

| Name: | | NBR 1 | NBR 2 | NBR 3 | NBR 4 | NBR 5 | NBR 6 |
|---|---|---|---|---|---|---|---|
| Formulation: | | | | | | | |
| acrylonitrile | phm | 37.6 | 37.4 | 37.4 | 37.4 | 37.6 | 37.6 |
| butadiene | phm | 62.4 | 62.6 | 62.6 | 62.6 | 62.4 | 62.4 |
| monochlorobenzene | phm | 59.1 | 58.9 | 59.2 | 59.2 | 59.1 | — |
| acetone | phm | — | — | — | — | — | 42.4 |
| Vazo ® 88 | phm | 0.13 | 0.084 | 0.017 | 0.0084 | 0.050 | 0.27 |
| tetrazole-RAFT | phm | 2.7 | 3.6 | 0.44 | 0.11 | 1.1 | 10.7 |
| Reaction conditions: | | | | | | | |
| Polymerization | ° C. | 100 | 100 | 100 | 100 | 100 | 100 |
| temperature | | | | | | | |
| Polymerization time | h | 5 | 5.5 | 8 | 22 | 7 | 3 |
| Final conversion | % | 18.0 | 18.5 | 10.8 | 8.9 | 12.0 | 37.2 |
| Analysis: | | | | | | | |
| Mₙ | kg/mol | 6.2 | 3.6 | 18 | 75 | 9.3 | 1.2 |
| M_{w} | kg/mol | 8.3 | 4.4 | 27 | 121 | 13 | 1.4 |
| PDI | | 1.3 | 1.2 | 1.5 | 1.6 | 1.4 | 1.2 |

### Example 3

### Coupling of Tetrozole-Functional NBRs

The coupling of the nitrile rubbers was effected according to the base formulation specified under the conditions specified in Table 2.

In a typical coupling reaction, tetrazole-functional NBR was dissolved in the solvent specified and the appropriate amount of a stock solution of 1,6-bis(maleimido)hexane was added. The reaction mixtures were irradiated with UV light of wavelength 254 nm at room temperature under air for 2-3 hours while stirring. The coupled polymers were obtained by removing the solvent under reduced pressure.

TABLE 2
Coupling of tetrazole-functional NBRs

| Name: | | NBR 7 | NBR 8 | NBR 9 | NBR 10 | NBR 11 | NBR 12 |
|---|---|---|---|---|---|---|---|
| Formulation: | | | | | | | |
| tetrazole-NBR | No. | NBR1 | NBR2 | NBR3 | NBR4 | NBR1 | NBR1 |
| | mg | 30.0 | 40.0 | 40.0 | 120 | 30 | 30 |
| 1,6-bis(maleimido)hexane | mg | 1.02 | 2.10 | 0.44 | 0.33 | 1.02 | 1.02 |
| acetonitrile | ml | 50 | 6 | 6 | 6 | — | — |
| methylene chloride | ml | — | — | — | — | — | 50 |
| chlorobenzene | ml | — | — | — | — | 50 | — |
| Reaction conditions: | | | | | | | |
| Reaction temperature | ° C. | 25 | 25 | 25 | 25 | 25 | 25 |
| Reaction time | min | 120 | 180 | 180 | 180 | 120 | 120 |
| Analysis: | | | | | | | |
| Mₙ | kg/mol | 8.9 | 6.5 | 35 | 94 | 8.4 | 9.2 |
| M_{w} | kg/mol | 14 | 8.5 | 51 | 150 | 13 | 16 |
| PDI | | 1.6 | 1.3 | 1.5 | 1.6 | 1.5 | 1.7 |

What is claimed is:

1. A process for producing a nitrile rubber coupled via bisdihydropyrazole groups comprising reacting a nitrile rubber which is based on conjugated dienes, α,β-unsaturated nitriles, and no, one or more further copolymerizable monomers as monomers, and has covalently bonded tetrazole groups with a bifunctional ene compound in which the ene groups are each reacted with the tetrazole groups to give dihydropyrazole groups.

2. The process according to claim 1, wherein the tetrazole group takes the form of a radical of the general formula (1) 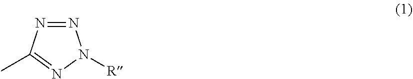
in which R″ is an aryl radical or a substituted an radical.

3. The process according to any one of claims 1 to 3, comprising reacting a nitrile rubber which has been hydrogenated.

4. The process according to any one of claims 2-3, wherein the nitrile rubber to be coupled is obtainable by free-radical polymerization of at least one conjugated diene, at least one unsaturated nitrite and no, one or more further copolymerizable monomers, in the presence of at least one organic solvent and at least one chain transfer agent, the chain transfer agent used being at least one compound of the general formula (I) 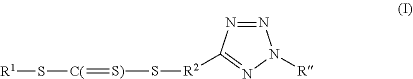
where
R¹ is a hydrocarbyl radical which after homolytic scission of the R¹—S bond, forms a primary radical,
R² is a hydrocarbyl radical which after homolytic scission of the S—R² bond, forms a secondary, tertiary or aromatically stabilized radical,
R″ is an aromatic radical or a substituted aromatic radical.

5. The process according to claim 4, wherein the compound of the general formula (I). R is a C₈-C₁₅-alkyl radical and R² is a —CHR³—C(═O)OR⁴— radical where R³ is C₁-C₃-alkyl and R⁴ is C₁-C₆-alkylene, and R″ is phenyl, tolyl, naphthyl or anthracenyl.

6. The process according to claim 5, wherein R⁴ is C₁-C₆-alkylene which is interrupted by (i) carboxyl groups, or (ii) aryl groups or (iii) carboxyl and aryl groups.

7. The process according to any of claims 1 to 6, wherein the bifunctional ene compound, two ene groups of the general formula (II)
R′—X—CH═CH—X—R′ (II)
are present, in which each X is independently a C═O, CHOH, CHI, CHBr, CHCl, CHNO₂, CHNH₂, CHCOOH, CHC₆H_{S}, CHCN radical and each R′ is independently a C₁-C₁₂-hydrocarbyl radical which may contain one or more heteroatoms, where the two ene groups of the general formula (II) are covalently bonded to one another via at least one of the R′ radicals in each case or in the alternative either in one or in both ene groups the two R′ radicals jointly form a radical Y which represents a C₂-C₂₄-hydrocarbyl radical which may contain one or more heteroatoms, thereby forming a ring together with the adjacent unit —X CH═CH—X— and wherein the link to the second ene group is then effected by a third coordination valence of the radical Y.

8. The process according to claim 7, wherein the bifunctional ene compound has two maleimide groups wherein the nitrogen atoms are joined to one another via a C₁-C₁₂-alkyl radical which is either unsubstituted or substituted.

9. The process according to claim 8, wherein the C₁-C₁₂-alkyl radical is interrupted by (i) heteroatoms, or (ii) aryl groups or (iii) heteroatoms and aryl groups.

10. The process according to any one of claims 1 to 3, wherein the nitrile rubber to be coupled is obtainable by
a) free-radical polymerization of at least one conjugated diene, at least one α,β-unsaturated nitrile and no, one or more further copolymerizable monomers in the presence of at least one chain transfer agent, and
wherein the chain transfer agent used in step a) is at least one compound of the general structural formula (VI) 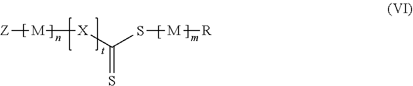
in which
Z is H, a linear or branched, saturated or mono- or polyunsaturated alkyl radical, a saturated or mono- or polyunsaturated carbo- or heterocyclyl radical, aryl, heteroaryl, aralkyl, heteroaralkyl, alkoxy, aryloxy, heteroaryloxy, amino, amido, hydroxyimino, carbamoyl, alkoxycarbonyl, F, Cl, Br, I, hydroxyl, phosphonato, phosphinato, alkylthio, arylthio, sulphanyl, thiocarboxyl, sulphinyl, sulphono, sulphino, sulpheno, sulphonic acids, sulphamoyl, silyl, silyloxy, nitrile, carbonyl, carboxyl, oxycarbonyl, oxysulphonyl, oxo, thioxo, borates, selenates, epoxy, cyanates, thiocyanates, isocyanates, thioisocyanates and isocyanides,
R (a) if m≠0 has the same meanings as the Z radical and
(b) if m=0 is H, a linear or branched, saturated or mono- or polyunsaturated alkyl radical, a saturated or mono- or polyunsaturated carbo- or heterocyclyl radical, aryl, heteroaryl, aralkyl, heteroaralkyl, alkoxy, aryloxy, heteroaryloxy, amino, amido, carbatnoyl, alkoxy, aryloxy, alkylthio, aryithio, sulphanyl, thiocarboxyl, sulphinyl, sulphono, sulphino, sulpheno, sulphonic acids, sulphamoyl, carbonyl, carboxyl, oxycarbonyl, oxysulphonyl, oxo, thioxo, epoxy, cyanates, thiocyanates, isocyanates, thioisocyanates or i socyani des,
M represents repeat units of one or more mono- or polyunsaturated monomers comprising conjugated or non-conjugated dienes, alkynes and vinyl compounds, or a structural element which derives from polymers comprising polyethers, especially polyalkylene glycol ethers and polyalkylene oxides, polysiloxanes, polyols, polycarbonates, polyurethanes, polyisocyanates, polysaccharides, polyesters and polyamides,
n and m are the same or different and are each in the range from 0 to 10 000,
t is 0 or 1 if n=0, and is 1 if n 0, and
X is C(Z₂), N(Z), P(Z), P(═O)(Z), O, S, or S(═O)₂, where Z in these radicals has the same meanings as stated above for the formula (VI),
and subsequent reaction of the nitrile rubber thus obtained with a compound which allows covalent attachment of a tetrazole group to the nitrile rubber and leads to this attachment.

11. The process according to claim 10, wherein the reaction step a) is followed by a hydrogenation as step h).

12. A nitrile rubber coupled via bisdihydropyrazole groups.

13. The nitrile rubber according to claim 12, obtainable by a process according to any of claims 1 to 11.

14. A process for the production of mouldings, coatings or vulcanizates comprising subjecting the nitrile rubbers coupled via bisdihydropyrazole groups according to claim 12 or 13 to the formation of a moulding, coating or vulcanizate.

15. A vulcanizable mixture comprising the nitrile rubber according to claim 12 or 13, at least one crosslinker.

16. The vulcanizable mixture according to claim 15 additionally comprising at least one filler and one or more further rubber additives.

17. A process for producing vulcanizates, wherein the vulcanizable mixture according to claim 15 is subjected to crosslinking.

18. The process according to claim 17 wherein the crosslinking is performed by addition of at least one crosslinker or by photochemical activation.

19. A vulcanizate obtainable by the process according to claim 18.

20. The vulcanizate according to claim 19 representing mouldings.

21. A chain transfer agent of the general formula (I) 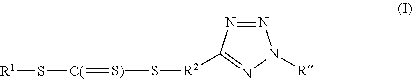
where
R¹ is a hydrocarbyl radical which after homolytic scission of the R¹—S bond, forms a primary radical,
R² is a hydrocarbyl radical which after homolytic scission of the S—R² bond, forms a secondary, tertiary or aromatically stabilized radical,
R″ is an aromatic radical or a substituted aromatic radical.

22. The chain transfer agent according to claim 21, wherein le is a C₈-C₁₅-alkyl radical and R² is a —CHR³—C(═O)OR⁴— radical where R³ is C₁-C₃-alkyl and R⁴ is C₁-C₆alkylene, and R″ is phenyl, tolyl, naphthyl or anthracenyl.
